(19) United States
(12) Patent Application Publication
(10) Pub. No.: US 2003/0228621 A1
(43) Pub. Date: Dec. 11, 2003

(54) Common ligand universal enzyme assay and compositions for use therein

(76) Inventors: Yong Qin, Poway, CA (US); Lin Yu, San Diego, CA (US); Mark R. Hansen, San Diego, CA (US); Eduard Sergienko, San Diego, CA (US); Bonnie Bertolaet, San Diego, CA (US); Daniel S. Sem, New Berlin, WI (US)

Correspondence Address: CAMPBELL & FLORES LLP, 7th Floor, 4370 La Jolla Village Drive, San Diego, CA, 92122, US

(21) Appl. No.: 10/189,327

(22) Filed: Jul. 2, 2002

Related U.S. Application Data
(60) Provisional application No. 60/383,448, filed on May 24, 2002.

(51) Int. Cl. G01N 33/53; C12Q 1/26

(52) U.S. Cl. 435/7.1; 435/25

(57) ABSTRACT

The present invention provides compositions containing a common ligand linked to a detectable moiety and provides methods for the preparation of such compositions. The present invention also provides methods for screening candidate ligands for binding to a NAD binding receptor, which include contacting a receptor with a candidate ligand and a composition of the invention followed by evaluation of receptor binding. The screening method of the present invention has broad applicability and can be used to screen large numbers of a wide variety of ligands. The present invention further provides methods for detecting the binding activity of a putative receptor, which include combining the putative receptor with a composition of the invention and evaluating the level of detectable moiety. The invention also provides kits useful for detection of receptors having NAD binding activity and for screening of candidate ligands that bind to a NAD binding receptor.

### RELATED APPLICATIONS

This application claims the benefit of U.S. Provisional Application No. 60/383,448, filed May 24, 2002, which is incorporated herein by reference.

### BACKGROUND OF THE INVENTION

The present invention relates generally to drug discovery and more specifically to reporter molecules and ligand binding assays.

Drug discovery and development has been based on screening for lead compounds or, alternatively, on structure-based drug design. Screening for lead compounds involves generating a pool of candidate compounds. The candidate compounds are screened with a drug target of interest to identify lead compounds. Structure based drug design uses three-dimensional structural data of the drug target as a template to model compounds that inhibit critical residues that are required for activity of the drug target. Compounds identified as potential drug candidates are used as lead compounds for the development of candidate drugs that exhibit a desired activity toward the drug target. Drug targets can include receptors and enzymes. However, the screening processes to identify lead compounds or candidate drugs that bind to these targets can be laborious and time consuming.

Many bioanalytical screening processes are based on the oxidative status of nicotinamide adenine dinucleotide (NAD) or nicotinamide adenine dinucleotide phosphate (NADP). Many oxidoreductase enzymes can use these cofactors to transfer hydrogen groups between molecules. Because the reduced forms of these molecules differ from their oxidized forms in their ability to absorb light, reactions have been quantitated based on light absorption at 340 nm or by fluorescent emission of light at 445 nm. Reduced nicotinamide adenine dinucleotide (NADH) is fluorescent, whereas NAD is not. Accordingly, enzymatic reactions based on NAD and NADH are amenable to fluorescent analysis.

NAD and NADP can be reversibly reduced by the addition of hydride ions. While both molecules can act as coenzymes in reversible reactions, NAD has been generally used as an acceptor of reducing equivalents in catabolism, while NADH is reoxidized by complex I of the electron transport chain or by dehydrogenase enzymes during anaerobic metabolism. NADPH is characteristically involved with reductive synthesis reactions, such as fatty acid synthesis.

NAD has a multiple ringed structure, which undergoes redox reactions within its nicotinamide ring. The closely related NADP molecule is phosphorylated on the 2′ position of the adenosine ribose ring. A redox reaction, such as the conversion of lactate to pyruvate by the enzyme lactate dehydrogenase requires the reduction of equimolar amounts of NAD to NADH.

Enzymatic dehydrogenase reactions can take advantage of the property of the reduced forms of NAD and NADP to absorb light at a wavelength of 340 nm while the oxidized form does not. Similarly, the reduced forms are capable of fluorescent emission at 445 nm when excited at 340 nm, while the oxidized forms are not. These properties permit quantitation of reactions that directly involve a change in the oxidative state of these cofactors. For example, when phosphoglycerate kinase and glyceraldehyde-3-phosphate dehydrogenase are used to catalyze the formation of NAD from NADH in the presence of adenosine triphospate (ATP), the concentration of adenosine triphosphate can be measured as a decrease in fluorescence intensity (U.S. Pat. Nos. 4,446,231 and 4,735,897).

Bioanalytical screening assays require the conversion of the oxidized forms of NAD and NADP to the reduced forms or of the reduced forms of NAD and NADP to the oxidized forms. It would be advantageous to have a reporter molecule which exhibits a change in fluorescence upon binding to an oxidoreductase, without the need for catalytic activity of the oxidoreductase to oxidize or reduce the co-factor.

Fluorescein-labeled substrates have been used in enzyme assays using fluorescence intensity or fluorescence polarization technology. For example, protease assays using a fluorescein-tagged protein substrate were developed by Spencer et al., Clin. Chem. 19:838-844 (1973); Maeda et al., Anal. Biochem., 92:222-227 (1979); and Sem & McNeeley, Febs. Lett. 443:17-19 (1999).

In order for a reporter molecule to be practically useful in a screening assay, there is a need for the reporter molecule to be sensitive, to have specificity for one or more receptors and to provide a strong signal for quantitative determination of ligand binding. Heretofore, there has not been a reporter molecule having these properties for direct, specific and sensitive screening of ligands which bind to a receptor which binds to a NAD cofactor and for the detection of the binding activity of a receptor which binds to a NAD cofactor, in the absence of catalysis.

Thus, there exists a need for a reporter molecule that is highly sensitive and specific for a receptor which binds to a NAD cofactor and which provides a strong signal upon binding to the receptor. There further exists a continuing need for the development of more sensitive and rapid methods for identification of ligands that bind to a receptor drug target which binds to a NAD cofactor. There further exists a need for high throughput screening of oxidoreductase ligand libraries and for secondary assays of oxidoreductase drug ligands. In addition, there exists a need to rapidly identify oxidoreductase binding activity corresponding to genes newly identified from genomic studies. There also exists a need for improved methods of synthesis of reporter molecules that provide a strong signal upon binding to a receptor which binds to a NAD cofactor. The present invention satisfies these needs and provides related advantages as well.

### SUMMARY OF THE INVENTION

The present invention provides compositions comprising a common ligand and methods for preparing such compositions. The present invention also provides methods for screening candidate ligands for binding to a NAD binding receptor and methods for detecting the binding activity of a putative NAD binding receptor. The present further invention provides kits for the screening of candidate ligands and for the identification of putative receptors having NAD binding activity.

In one aspect, the present invention provides compositions containing a common ligand, such as an oxidoreductase common ligand. More particularly, the compositions of the invention can comprise, for example, a NAD common ligand linked to a detectable moiety, a dye common ligand linked to a detectable moiety, or a dye common ligand. The compositions of the invention are detectable by conventional detection systems and are useful for determining NAD binding activity of putative receptors and for screening candidate ligands for binding to a NAD binding receptor, such as an oxidoreductase.

In a second aspect, the present invention provides methods for preparing the compositions of the invention. In general, such methods include reacting a common ligand with a detectable moiety followed by purification of the resultant composition. In one embodiment, the common ligand can be derivatized with one or more linking groups or functional groups prior to reaction with the detectable moiety. In another embodiment, the resultant composition can be reduced, followed by purification of the reduced product.

In another aspect, the present invention provides methods for detecting NAD binding activity. In one embodiment, the present invention provides methods for detecting NAD binding activity of a putative NAD binding receptor. Generally, these methods involve contacting a putative receptor with a composition of the invention and detecting binding activity. The presence of binding activity indicates that the putative receptor is a NAD binding receptor as defined herein. In another embodiment, these methods can be used to screen for NAD binding activity of a population of putative NAD binding receptors.

In another embodiment, the present invention provides methods for screening candidate ligands for NAD binding activity. Generally, the methods involve contacting a NAD binding receptor with a composition of the invention to form a composition:NAD binding receptor complex, followed by contact of the complex with the candidate ligand. Binding activity of the candidate ligand is determined by measuring displacement of the composition by the candidate ligand. Alternatively, a NAD binding receptor can be contacted with a candidate ligand to form a candidate ligand:NAD binding receptor complex, followed by contact of the complex with a composition of the invention. Binding activity of the candidate ligand is determined by measuring signal from the composition to determine the amount of the candidate ligand displaced by the composition.

In a further embodiment, the present invention provides methods for screening candidate ligands for NAD binding activity which involve contacting a NAD binding receptor, a candidate ligand, and a composition of the invention and measuring binding activity.

The screening methods of the present invention have broad applicability and can be used to screen large numbers of a wide variety of ligands. In one embodiment of the invention, the methods are used to screen candidate ligands for an oxidoreductase receptor, such as a receptor for alcohol dehydrogenase, dihydrodipicolinate reductase, enoyl ACP reductase, glyceraldehyde-3-phosphate dehydrogenase, or lactate dehydrogenase. In another embodiment, the present invention is used to screen populations of candidate ligands for NAD binding activity.

The invention further provides kits useful for detection of NAD binding receptors or for the detection of candidate ligands that bind to a NAD binding receptor. Such kits contain a detectable composition of the invention. Optionally, the kit also can contain a NAD binding receptor.

### BRIEF DESCRIPTION OF THE DRAWINGS

FIG. 1 shows a reaction pathway for the synthesis of fluorescent NADH and reduced nicotinamide adenine dinucleotide phosphate (NADPH).

FIG. 2 shows the binding curves of FITC-NADH to alcohol dehydrogenase (ADH), dihydrodipicolinate reductase (DHPR), enoyl ACP reductase (EACPR), glyceraldehyde-3-phosphate dehydrogenase (GAPDH) and lactate dehydrogenase (LDH).

FIG. 3 shows the binding curve of FITC-NADH to dihydrodipicolinate reductase (DHPR) at varying concentrations of FITC-NADH.

FIG. 4 shows the binding curve of FITC-NADH titrated with dihydrodipicolinate reductase (DHPR) at 25 nM FITC-NADH.

FIG. 5 shows the binding curve of FITC-NADH titrated with enoyl ACP reductase (EACPR) at 1.5 nM FITC-NADH.

FIG. 6 shows stability curves of the dehydrogenases enoyl ACP reductase, dihydrodipicolinate reductase and glyceraldehyde-3-phosphate dehydrogenase with FITC-NADH plotted as time in minutes versus Polarization, mP.

FIG. 7 shows the change in fluorescence polarization when NADH displaces FITC-NADH bound to dihydrodipicolinate reductase (DHPR).

FIG. 8 shows the change in fluorescence polarization when NADH displaces FITC-NADH bound to enoyl ACP reductase (EACPR).

FIG. 9 shows the change in fluorescence polarization when an inhibitor bromaminic acid displaces FITC-NADH bound to dihydrodipicolinate reductase (DHPR), at 7 μM DHPR, 25nM FITC-NADH. Error bars are shown for each measurement indicating the variance in results for 3 experiments.

FIG. 10 depicts an assay scheme to determine the reversibility of enzyme inhibition.

FIG. 11 shows the structures of three triazinyl dyes that inhibited most of the dehydrogenases tested.

FIGS. 12A and 12B show inhibition of DOXPR enzymatic activity by RR120 and RG5.

FIG. 13 shows a schematic representation of the displacement assay between dyes of the invention and FITC-NADPH label.

FIGS. 14A and 14B show the results of a displacement assay between either Reactive Red 120 or Reactive Green 5 and FITC-NADPH for binding to DOXPR.

FIGS. 15A to 15C show the structures of exemplary triazinyl dyes which are useful as NAD cofactors in the present invention.

FIGS. 16A through 16G depict the structures of several potent oxidoreductase common ligands of the present invention which have binding affinity for DHPR and DOXPR.

### DETAILED DESCRIPTION OF THE INVENTION

The present invention provides compositions and methods for assaying NAD binding activity. These compositions and methods are universally applicable for determining NAD binding activity of a variety of receptors.

The compositions of the invention are based on mimics of NAD that can bind to the NAD binding site of a receptor. Based on their ability to bind to an NAD binding site, the compositions can be used to determine NAD binding activity of receptors, in particular, members of the oxidoreductase receptor family which bind NAD and/or NADP. The compositions, therefore, can be used to determine NAD binding activity of a receptor using the sensitive screening assays of the invention.

The ability of the compositions to detect NAD binding in sensitive assays provides several advantages over conventional screening assays. For example, newly identified genes from genomics studies can be screened for NAD binding activity without the need to determine a substrate for measuring enzyme activity, as would be required when using activity-based assays and the natural ligand NAD or NADP. Thus, the compositions and methods of the invention can be readily applied to rapidly determine NAD binding activity of newly identified genes from genomics studies.

Furthermore, the ability of the compositions of the invention to bind to a NAD binding site allows screening of compounds for NAD binding activity based on competitive binding with the compositions. Thus, the compositions and methods of the invention can be used to rapidly and efficiently screen libraries of potential ligands for binding activity for a receptor, which can be used as drug candidates or lead compounds for further drug discovery.

In a first aspect, the present invention provides compositions having binding activity for a NAD binding site on a receptor. The compositions can be used in methods of the invention for assaying NAD binding activity. The compositions of the invention allow efficient, high throughput screening of large numbers of molecules for binding to a receptor which also binds to a NAD.

The compositions of the invention comprise a common ligand, for example, an oxidoreductase common ligand. As used herein, a “common ligand” when used in reference to binding activity for a NAD/NADP binding receptor is a compound that selectively binds to a NAD binding site on such a receptor. Thus, a common ligand is any molecule which binds to a NAD binding site of a receptor. Nonlimiting examples of receptors having NAD binding sites include oxidoreductases, catalases, epimerases, ADP ribosylase, synthases, and cyclases.

Common ligands of the invention include, for example, oxidoreductase common ligands. As used herein, an “oxidoreductase common ligand” refers to a compound that selectively binds to a NAD binding site of an oxidoreductase. As used herein, the term “NAD binding site” refers to a site on a receptor that binds NAD, NADP, NADH, or NADPH. The term “selectively” as used herein refers to a binding interaction that is detectable over non-specific interactions by a quantifiable assay. It is understood that the oxidoreductase common ligand need not bind to all oxidoreductase receptors but does bind at least two oxidoreductases and can bind several, most, or all oxidoreductases.

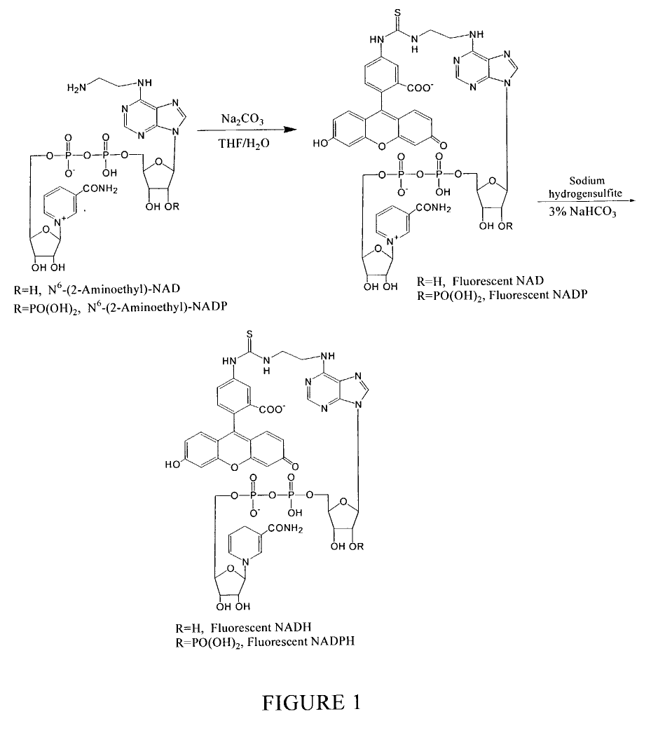

Common ligands also include, for example, NAD common ligands as discussed below. As used herein, “NAD common ligand” refers to NAD, NADH, NADP, or NADPH; to an analog of NAD, NADH, NADP, or NADPH that retains the ability to selectively bind to a NAD binding site on a receptor, such as an oxidoreductase or epimerase; or to a mimetic of NAD, NADH, NADP, or NADPH. As used herein, the term “analog” of NAD, NADH, NADP, or NADPH refers to a molecule that is structurally similar to NAD, NADH, NADP, or NADPH, and that retains the multiple ringed structure set forth in FIG. 1. Such analogs have, at least, a nicotinamide ring and an adenosine ribose ring as found in the parent molecule. Such analogs are known in the art and are described, for example, in Everse et al. (eds.) The Pyridine Nucleotide Coenzymes, Chap. 4 “Analogs of Pyridine Nucleotide Coenzymes, pp. 91-133, Academic Press (1982).

As used herein, a “mimetic” is any organic structure that exhibits structural and/or functional properties of the reference common ligand. Such structural properties include, for example, charge and charge spacing. For example, an organic structure which mimics NADH would have a negative charge moiety located in a molecular space similar to that of the pyrophosphate of the naturally occurring NADH. Such functional properties include, for example, binding properties. A mimetic can also be an isostere having an electronic configuration similar to the electronic configuration of NAD, NADH, NADP, or NADPH (see, for example, U.S. Pat. No. 5,658,890, incorporated by reference herein).

The NAD common ligand can be a naturally occurring molecule or a synthetic analog. Exemplary naturally occurring NAD common ligands include NAD, NADH, NADP, and NADPH. Synthetic analogs include, for example, mimetics. Other analogs are known in the art and are described, for example, in Everse et al., supra.

A NAD common ligand can be chemically modified, for example, by reduction and/or phosphorylation or by replacement of one or more oxygen atoms with one or more sulfur atoms. A NAD common ligand also can be chemically modified by the addition of one or more reactive groups for coupling to another moiety. For example, NAD which has been halogenated at the 8 position to form nicotinamide-8-(2-carboxyethylthio)adenine dinucleotide, can be used for coupling NAD to a macromolecule such as a polymer; see, for example, U.S. Pat. No. 4,336,188, incorporated by reference herein. Additionally, a NAD common ligand can be modified by the attachment of a 2-aminoethyl group at N⁶ of the adenine ring of NAD as described for example in Buckman, A. F., Biocatalysis 1:173-186 (1987) also incorporated by reference. Modified NAD common ligands can further include catalytically caged cofactors which can bind to a receptor, such as an enzyme, but do not allow turnover prior to photolytic activation.

Nonlimiting examples of modified NAD common ligands include, but are not limited to, alkylated common ligands, methyl phosphonates, phosphorodithioates, pyruvate adducts, silyl ethers, sulfonates, phosphorothioates, ethylenedioxy ethers, thio-NAD, thio-NADH, thio-NADP, thio-NADPH, 6-(2-hydroxy-3-carboxy-propylamino)adenine dinucleotide, 1,6-dihydro NAD, acetylpyridine adenine dinucleotide, dihydro-NAD, nicotinamide hypoxanthine dinucleotide, pyridine aldehyde adenine dinucleotide, pyridine aldehyde hypoxanthine dinucleotide and phosphates thereof, alpha-carboxy-2-nitrobenzyl NADP, 1-(4,5,dimethoxy-2-nitrobenzyl NADP), and salts of NAD common ligands. See, for example, U.S. Pat. Nos. 4,008,363; 4,258,131; 4,411,995; 4,590,602; 5,480,982; 6,046,018; and 6,162,615; incorporated by reference herein. Those skilled in the art know or can determine through routine methods what structures constitute functionally equivalent naturally occurring or synthetic NAD cofactors.

Examples of mimetics to the common ligand NADH, for example cibacron blue, are described in Dye-Ligand Chromatography, Amicon Corp., Lexington, Mass. (1980). Numerous other examples of NADH-mimics, including useful modifications to obtain such mimics, are described in Everse et al. (eds.), The Pyridine Nucleotide Coenzymes, Academic Press, New York (1982). Particular mimetics include nicotinamide 2-aminopurine dinucleotide, nicotinamide 8-azidoadenine dinucleotide, nicotinamide 1-deazapurine dinucleotide, 3-aminopyridine adenine dinucleotide, 3-acetyl pyridine adenine dinucleotide, thiazole amide adenine dinucleotide, 3-diazoacetylpyridine adenine dinucleotide and 5-aminonicotinamide adenine dinucleotide. Particular mimetics can be identified and selected by ligand-displacement assays, for example using competitive binding assays with a known ligand as is well known in the art. Mimetic candidates can also be identified by searching databases of compounds for structural similarity with the common ligand or a mimetic. While modification of common ligands of the invention has been described in terms of NAD common ligands, it is understood by those of ordinary skill in the art that similar modifications, particularly the addition of functional groups, can be made to any of the common ligands of the invention.

Common ligands of the invention also include, for example, dye common ligands. As used herein, “dye common ligand” refers to a dye compound that contains a triazine ring, a phenylsulphonyl moiety, or both and which selectively binds to a NAD binding site on oxidoreductase receptors. Such dyes are known in the art and generally are employed in affinity chromatography. Nonlimiting examples of dye common ligands useful in the present invention are Reactive Red 120, Reactive Green 5, Reactive Green 19, Reactive Blue 2, Reactive Blue 4, Reactive Blue 72, Cibacron Blue 3GA, Reactive Orange 14, Reactive Brown 10, Reactive Yellow 3, Reactive Yellow 86, and Naphthol Yellow S. Structures of exemplary dye common ligands are provided in FIG. 15.

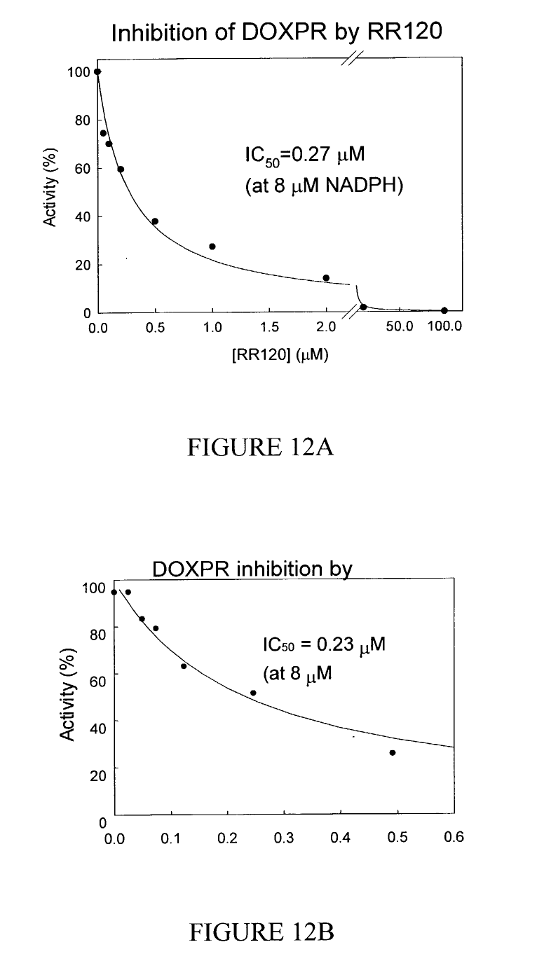

These dye common ligands are particularly useful for detection of ligands binding to the nucleotide binding site of oxidoreductases, in part, because they exhibit binding activity for multiple oxidoreductases and competitive binding with NAD/NADP. In one embodiment, the invention comprises the dye common ligands Reactive Green 5, Reactive Red 120, and Reactive Blue 2, each of which binds and inhibits the activity of a broad spectrum of oxidoreductases. Each of these dye common ligands exhibits full reversible inhibition of the enzymes, as shown, for example, for DOXPR in FIG. 12. The enzyme inhibition exhibited by the dye common ligand is a competitive inhibition versus cofactor substrate.

Although any dye common ligand can be employed in the present invention, Reactive Green 5 provides several advantages. In addition to binding a variety of oxidoreductases, Reactive Green 5 contains a chelated copper metal ion. Substitution of this metal ion with one that has fluorescence, for example ruthenium, rhenium, or osmium, results in a ligand that can be used in fluorescence resonance energy transfer (FRET). Similarly, substitution of the copper metal ion in Reactive Green 5 with a metal that has long-lifetime fluorescence, for example, lanthanides, such as europium, terbium, dysprosium, or samarium results in a ligand that can be used in time resolved fluorescence resonance energy transfer (Time Resolved FRET) assays for all oxidoreductases that bind Reactive Green 5.

Alternatively, the phthalocyanine moiety can be replaced with a commercially available cage for a lanthanide-metal ion, resulting in a Time-Resolved FRET tracer. To determine the implications of such a replacement on enzyme binding activity, the compound sulphophthalocyanine was tested for its inhibition of the dehydrogenases DHPR and DOXPR. These studies indicate that the phthalocyanine moiety interacts with dehydrogenases and, therefore, replacing the phthalocyanine moiety with a lanthanide cage may result in some loss of enzyme binding activity.

In one embodiment, the present invention comprises compositions containing an oxidoreductase common ligand linked to a detectable moiety. In these compositions, the oxidoreductase common ligand retains its ability to selectively bind to an oxidoreductase and additionally exhibits detectable binding over background binding by a quantifiable assay.

As used herein, a “detectable moiety” refers to a molecule that can be detected through physical or chemical means. Any detectable moiety can be employed in the present invention, so long as the detectable moiety does not interfere with the binding of the oxidoreductase common ligand to a receptor. For example, the detectable moiety can be a molecule detectable by analytical methods including, for example, fluorescent tags; fluorescent proteins, such as green fluorescent protein; radioactive tags; ferromagnetic substances; luminescent and chemiluminescent tags, chromophores and calorimetric indicators; detectable binding agents, such as members of a binding pair like biotin/streptavidin or antibodies/antigens.

One example of a detectable moiety useful in the present invention is a fluorescent tag. Fluorescent tags are well known in the art and are described, for example, in Hermanson, Bioconjugate Techniques, pp. 297-364, Academic Press, San Diego (1996). Fluorescent molecules useful in the invention include, but are not limited to, fluorescein and fluorescein derivatives; rhodamine and rhodamine derivatives; coumarin and coumarin derivatives; BODIPY™ (4,4-difluoro-4-bora-3a,4a-diaza-s-indacene) and BODIPY™ derivatives (Molecular Probes; Eugene, Oreg.); Cascade Blue™ and derivatives thereof (Molecular Probes); Lucifer Yellow (3,6-disulfonate-4-amino-napthalimide) and derivatives thereof; Alexa fluor dyes (Molecular Probes) and CyDye fluorescent dyes (Amersham Pharmacia Biotech; Piscataway, N.J.). Other non-limiting examples of fluorescent moieties include fluorescein isothiocyanate (FITC), eosins, erythrosins, Lissamine Rhodamine B, Oregon Green, Rhodamine Green, Rhodamine Red-X, Texas Red and related compounds, tetramethylrhodamine, and the like.

Another example of a detectable moiety useful in the present invention is a fluorescent protein. Fluorescent proteins include, but are not limited to, green fluorescent protein and derivatives thereof as well as phycobiliproteins, and derivatives thereof, such as phycoerythrin and phycocyanin. Nonlimiting examples of chromophores suitable as detectable moieties include phenolphthalein, malachite green, phytochromes and apophytochromes, yellow protein chromophore, melanophores, and phenanthrolines. Other colorimetric indicators, such as chemiluminescent molecules, which are useful as detectable moieties include, but are not limited to, 1,2-dioxetane and luminol. Nonlimiting examples radioactive tags suitable as detectable moieties include ³²P, ³³P, ³⁵S, ³H, ¹⁴C, ¹²⁵I, ⁵⁹Fe, and ¹⁸F.

The common ligand and detectable moiety can be linked to one another in any feasible manner. The linkage can be direct or via a linker molecule. For example, a fluorescent moiety can be covalently linked to a NAD common ligand or dye common ligand.

A detectable moiety of the invention can be linked to a common ligand via one or more linking groups. As used herein, the term “linking group” refers to a molecule having at least one functional group capable of reacting with a functional group on a common ligand, on a detectable moiety, or on a linking group attached to a common ligand or detectable moiety. As used herein, “functional group” refers to an atom or group of atoms that defines the structure of a particular family of organic compounds and determines their properties. Exemplary functional groups include, but are not limited to, amines, carboxylates, thiols, hydroxy groups, and the like.

Suitable linking groups for coupling the common ligand to the detectable moiety can be introduced onto the common ligand or the detectable moiety or both by chemical modification using conventional methods. In one embodiment of the invention, a linking group is introduced onto the adenine ring of a NAD common ligand. One suitable linking group for coupling to the adenine ring of a NAD common ligand is a 2-aminoethyl group. Other suitable linking groups include 3-aminopropyl, 4-aminobutyl, 5-aminopentyl, and the like.

The linking group can be coupled at any position on the oxidoreductase common ligand which permits attachment of the detectable moiety but does not impede binding of the labeled common ligand to a receptor. For example, a variety of different functionalities can be placed on the adenine ring of a NAD common ligand, and these analogs can still be recognized by a receptor such as an oxidoreductase. One suitable position for attachment of a functional group to a NAD common ligand is the N⁶ position on the adenine ring. Exemplary compositions in which a detectable moiety is coupled to a NAD common ligand via one or more chemically reactive functional groups include, for example, fluorescein isothiocyano-N⁶-(2-aminoethyl)-NAD, fluorescein isothiocyano-N⁶-(2-aminoethyl)-NADH, fluorescein isothiocyano-N⁶-(2-aminoethyl)-NADP, and fluorescein isothiocyano-N⁶-(2-aminoethyl)-NADPH.

Determination of those positions on the oxidoreductase common ligand to which a detectable moiety can be attached can be determined by a number of different methods well known to those skilled in the art. For example, structural models of NAD/NADP binding polypeptides with bound cofactor can be used to identify a position on the cofactor that is accessible to solvent and therefore available as an attachment site for a detect able moiety. Currently, over 200 crystal structures are available for oxidoreductases having a cofactor bound to them. Inspection of many of these structures, including those for dihydropicolinate reductase, alcohol dehydrogenase, and lactate dehydrogenase, was used to determine that the adenine portion of a NAD cofactor, specifically the exocyclic amino group of the adenine ring, is accessible to solvent and, therefore, is available for attachment of a detectable moiety. This can be determined by visual inspection of the structural complex generated by NMR; crystallography; computational docking as described, for example, in Doucet and Weber, Computer-Aided Molecular Design: Theory and Applications, Academic Press, San Diego, Calif. (1996); or by NMR-driven docking as described in copending U.S. patent application Ser. No. 10/158,770 filed May 30, 2002. Thus, it follows that the exocyclic amino group of the adenine ring of NAD common ligands of the present invention also are available for attachment of a detectable moiety.

Accessibility of a bound ligand to solvent also can be assessed by presenting the structural model of a cofactor-dehydrogenase complex as a surface representation and visually observing whether it is possible to see portions of the bound cofactor. The visible portions of the cofactor are accessible, and, therefore, are available to attach a detectable moiety. One skilled in the art would recognize that similar positions on the common ligands of the present invention also are available for attachment of a detectable moiety.

Accessibility further can be assessed by computationally rolling a water molecule over the surface of the enzyme-common ligand complex. If water can access the cofactor when the water molecule is rolled over the surface of the complex, the common ligand is accessible for attachment of a detectable moiety. Such methods are known in the art and are described, for example, in Doucet & Weber, supra.

Substituents can be attached to the exocyclic amino group on the adenine ring of a NAD common ligand without compromising binding affinity (see, for example, Everse et al, supra). This retention of binding affinity indicates that large chemical moieties can be attached to this amino group, and therefore this amino group is a good site for attachment of a detectable moiety.

Determination of solvent accessible regions as potential attachment points for a detectable moiety also can be determined on the basis of NMR structures of cofactor- or common ligand-enzyme complexes; see, for example, Pellecchia et al., J. Biomol. NMR 22: 165-173 (2002). NMR methods are useful because they avoid the need to determine complete three dimensional structures and, thus, provide a rapid means for determining which portions of a common ligand are buried and which portions are accessible to solvent. Those positions which are accessible are potential attachment points for a detectable moiety; see, for example, copending application Ser. No. 10/158,770 filed May 30, 2002.

Many of the dye common ligands of the invention are known affinity chromatography ligands. Positions for attachment of the detectable moiety to a dye common ligand can be determined on the basis of their function in affinity chromatography. These compounds bind affinity resins through reaction of a chloride ion attached to the triazinyl ring, which can react at an elevated pH with —OH, —SH, and NH₂ groups on the affinity resin. Binding studies show that resin bound dye common ligands retain binding affinity for oxidoreductases. These studies indicate that, in a dye common ligand-enzyme complex, the triazine ring on the dye common ligand is available for attachment of the detectable moiety.

As discussed above, a dye common ligand can be attached to an extrinsic detectable moiety. However, such dye common ligands can also be used without the addition of an extrinsic detectable moiety since the dye itself is detectable. In such instances, the dye common ligand can be used by itself as a detectable molecule that binds to an NAD/NADP binding site.

In another aspect, the present invention also provides methods for preparation of the compositions of the invention. Compositions comprising a common ligand and a detectable moiety can be prepared by methods known in the art that are suitable for the particular format of the detection method employed. The common ligand can be coupled directly to the detectable moiety, for example, through functional groups located on the common ligand and/or the detectable moiety. The common ligand and/or the detectable moiety also can be modified by conventional means to add one or more functional groups directly to the molecule to facilitate coupling.

Alternatively, the common ligand and detectable moiety can be coupled through the use of one or more linking groups. Either or both of the common ligand and the detectable moiety can be modified to contain a linking group. For example, the common ligand can be modified to contain a linking group having a functional group that will react either with a functional group directly attached to a detectable moiety or with a functional group on a linking group attached to a detectable moiety. Further, the common ligand and detectable moiety can be linked indirectly through a single linking group containing two functional groups, one that attaches to a functional group on the common ligand and the other that attaches to a functional group on the detectable moiety, where the functional groups on the common ligand and detectable moiety can be attached directly to the molecule or attached via a linking group.

As described in Example 1 below, a NAD common ligand can be derivatized at the N⁶ position of the adenine ring. In one embodiment of the invention, NAD or NADP can be derivatized with a 2-(aminoethyl) group to produce N⁶-(2-aminoethyl)-NAD or N⁶-(2-aminoethyl)-NADP, see, for example, Buckman, A. F., Biocatalysis 1:173-186 (1987). These derivatives then can be reacted with a detectable moiety that reacts with the 2-aminoethyl group to produce a detectable composition which retains binding activity to the enzyme.

In one embodiment of the invention, a composition comprising an oxidoreductase common ligand and a detectable moiety is prepared by reacting an oxidoreductase common ligand with a fluorescent moiety to form a detectable composition. The fluorescent composition is then purified. For example, the invention provides a method for the preparation of a fluorescent composition of NAD comprising reacting NAD with a fluorescent moiety. The resulting fluorescent NAD composition then can be purified using methods well known in the art. Similarly, the invention provides a method for the preparation of a fluorescent composition of NADP, comprising reacting NADP with a fluorescent moiety. The resulting fluorescent NADP composition then can be purified using methods well known in the art. While preparation of the invention is exemplified in terms of NAD common ligands and fluorescent moieties, other compositions of the invention can be prepared in a similar manner by reacting the particular oxidoreductase common ligand with a detectable moiety and purifying the resultant composition.

In another embodiment, the method of preparing a composition of the invention further can include reducing the detectable composition and subsequently purifying the composition. For example, where the composition comprises NAD as the common ligand and a fluorescent detectable moiety, the method can further include reducing fluorescent NAD or fluorescent NADP produced as described above. The reduced fluorescent NAD or fluorescent NADP can be subsequently purified using methods well known in the art.

In one specific embodiment, the methods for preparing a composition comprising a NAD common ligand can include a step of coupling the NAD common ligand at the N⁶ position of the adenine ring to a fluorescent moiety. The NAD common ligand can be coupled to any fluorescent moiety, so long as the fluorescent moiety does not interfere with the binding of the common ligand to the enzyme. Exemplary fluorescent moieties suitable in the methods of the invention include, but are not limited to, Alexa Fluor Dyes, BODIPY (4,4-difluoro-4-bora-3a,4a-diaza-5-indacene), Cascade Blue, fluorescein isothiocyanate (FITC), eosins, erythrosins, Lissamine Rhodamine B, Oregon Green, Texas Red and related compounds, Rhodamine Green, Rhodamine Red-X, tetramethylrhodamine, and the like.

In another specific embodiment for preparing a composition of the invention, the fluorescent moiety can be a derivative of fluorescein, for example, fluorescein isothiocyanate. The fluorescein isothiocyanate can be coupled to the common ligand via any suitable functional group, as previously described herein, which can be introduced, for example, by chemical modification of the common ligand. One such suitable functional group is a 2-aminoethyl group. For example, the method can be employed to prepare fluorescein isothiocyano-N⁶-(2-aminoethyl)-NAD, fluorescein isothiocyano-N⁶-(2-aminoethyl)-NADH, fluorescein isothiocyano-N⁶-(2-aminoethyl)-NADP, or fluorescein isothiocyano-N⁶-(2-aminoethyl)-NADPH. Although specific embodiments of the invention have been described with regard to the formation of compositions comprising NAD common ligands and fluorescent detectable moieties, compositions of the invention comprising other common ligands and other types of detectable moieties can be prepared using similar methods. Modification of the disclosed method to prepare additional compositions of the invention is within the level of skill of the ordinary artisan in view of the disclosure herein.

In another aspect, the present invention provides methods for detection of NAD binding activity. The detection methods can be qualitative or quantitative and can be performed using well known methods. For example, the methods of the invention can be used to determine the binding activity of a polypeptide to compositions of the invention to determine whether the polypeptide has NAD binding activity and therefore whether the compound is a NAD binding receptor as defined herein. The methods of the invention also can be used to screen candidate ligands for competitive binding with the compositions of the invention to determine the ability of such ligands to bind a NAD binding receptor.

As used herein, the term “NAD binding receptor” refers to a polypeptide, that has selective binding affinity for a naturally occurring NAD cofactor. Naturally occurring NAD cofactors are well known in the art and include NAD, NADH, NADP, and NADPH. NAD binding receptors include, for example, oxidoreductases, catalases, epimerases, ADP ribosylases, synthases, and cyclases. Thus, the present invention encompasses common ligands of such NAD binding receptors. For example, the invention includes, but is not limited to catalase common ligands, epimerase common ligands, ADP ribosylase common ligands, sythase common ligands, and cyclase common ligands. As used herein, each of these terms refer to a compound that selectively binds to a NAD binding site of the named enzyme. For example an epimerase common ligand is a compound that selectively binds to a NAD binding site of an epimerase.

The dissociation constant of the NAD binding receptor for a NAD cofactor will generally be less than about 10⁻⁴ M, for example less than 10⁻⁵ M, and less than 10⁻⁶ M, including less than about 10⁻⁸ M and less than about 10⁻⁹ M. An NAD binding receptor also can be a partially or completely synthetic derivative or analog of such a polypeptide or a sequence mutation of a naturally occurring NAD binding receptor, including insertions, deletions, and substitutions, of such a peptide so long as the NAD binding receptor exhibits selective binding to a NAD cofactor.

Although it is not necessary for the NAD binding receptor of the invention to have catalytic activity, the NAD binding receptor can also be, for example, an enzyme that carries out a catalytic reaction, converting a substrate to a product utilizing a NAD cofactor. For example, an NAD binding receptor can be an oxidoreductase that carries out a catalytic reaction converting a substrate to a product utilizing a NAD cofactor. Alternatively, the NAD binding receptor can be an enzyme, such as a cyclase, that carries out a catalytic reaction which directly utilizes a NAD cofactor as a substrate. The NAD binding receptor also can be an enzyme, such as a synthase, that produces a NAD cofactor as a product. Further, the NAD binding receptor can be an enzyme that catalyzes the conversion of a NAD cofactor to a product. For example, both ADP ribosyl cyclase and NAD glycohydrolase catalyze the conversion of beta-NAD to a cyclized ADP-ribose having an N-glycosyl linkage between the anomeric carbon of terminal ribose and the N⁶-amino group of the adenine moiety; see, for example, U.S. Pat. No. 5,608,047 incorporated by reference herein. The NAD binding receptor can also catalyze the synthesis of a NAD cofactor. For example, the conversion of nicotinamide mononucleotide to NAD is catalyzed by nicotinamide mononucleotide adenyltransferase, and the conversion of nicotinic acid mononucleotide NAD is catalyzed by the action of NAD synthase.

An NAD binding receptor of the present invention can be determined by the presence of a recognizable protein motif which indicates that the compound is a dehydrogenase. The binding motif for dehydrogenases is GXXGXXG or GXGXXG, a hydrophobic core of six small hydrophobic residues, a conserved, negatively charged residue that binds to the ribose 2′ hydroxyl of adenine and conserved positively charged residue. (Rossman et al., in The Enzymes Vol. 11, Part A, 3^{rd} ed., Boyer ed., pp. 61-102, Academic Press, New York(1975); Wiegrena et al. J. Mol. Biol. 187:101-107 (1986); Ballamacina, FASEB J. 10:1257-1269 (1996) Accordingly, the presence of an amino acid motif specific for an oxidoreductase can be used to characterize a newly identified gene as a putative oxidoreductase. The compositions and methods of the present invention can be advantageously used to determine that the putative oxidoreductase has NAD/NADP binding activity to confirm that the putative oxidoreductase is in fact an oxidoreductase.

Enzymes can be classified based on Enzyme Commission (EC) nomenclature recommended by the Nomenclature Committee of the International Union of Biochemistry and Molecular Biology (IUBMB)(see, for example, www.expasy.ch/sprot/enzyme.html). Oxidoreductase enzymes utilize NADH or NADPH as cofactors. For example, oxidoreductases are classified as oxidoreductases acting on the CH—OH group of donors with NADH or NADPH as an acceptor (EC 1.1.1); oxidoreductases acting on the aldehyde or oxo group of donors with NADH or NADPH as an acceptor (EC 1.2.1); oxidoreductases acting on the CH—CH group of donors with NADH or NADPH as an acceptor (EC 1.3.1); oxidoreductases acting on the CH—NH₂ group of donors with NADH or NADPH as an acceptor (EC 1.4.1); oxidoreductases acting on the CH—NH group of donors with NADH or NADPH as an acceptor (EC 1.5.1); oxidoreductases acting on NADH or NADPH (EC 1.6); and oxidoreductases acting on NADH or NADPH with NADH or NADPH as an acceptor (EC 1.6.1).

Additional oxidoreductases include oxidoreductases acting on a sulfur group of donors with NADH or NADPH as an acceptor (EC 1.8.1); oxidoreductases acting on diphenols and related substances as donors with NADH or NADPH as an acceptor (EC 1.10.1); oxidoreductases acting on hydrogen as donor with NADH or NADPH as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NADH or NADPH as an acceptor (EC 1.16.1); oxidoreductases acting on —CH₂ groups with NADH or NADPH as an acceptor (EC 1.17.1); and oxidoreductases acting on reduced ferredoxin as donor, with NADH or NADPH as an acceptor (EC 1.18.1). In addition, newly identified oxidoreductases that bind a NAD cofactor are within the scope of the invention.

Any oxidoreductase that is a NAD binding receptor is suitable for use in the present invention. Exemplary oxidoreductases include adenosylhomocysteine hydrolase, L-alanine dehydrogenase, alcohol dehydrogenase (ADH), aldose reductase (AR), catalase, 1-deoxy-D-xylulose 5-phosphate reductoisomerase (DOXPR), dihydrodipicolinate reductase (DHPR), dihydrofolate reductase (DHFR), 3-isopropylmalate (IPMDH), enoyl ACP reductase (EACPR), formate dehydrogenase, glyceraldehyde-3-phosphate dehydrogenase (GAPDH), D2-hydroxyisocaproate dehydrogenase, 3-hydroxy-3-methylglutaryl coenzyme A reductase (HMG COAR), inosine monophosphate dehydrogenase (IMPDH), lactate dehydrogenase (LDH), malate dehydrogenase, P450 reductase, D3-phosphoglycerate dehydrogenase, shikimate dehydrogenase, tetrahydrofolate reductase, trypanothione reductase, and steroid dehydrogenases.

As used herein, the term “candidate ligand” is intended to mean a molecule that potentially can bind specifically with a receptor as defined herein. A candidate ligand can be a known or unknown molecule. A candidate ligand can be a naturally occurring molecule or a synthetic analog of a ligand known to bind to a receptor. A candidate ligand which is a synthetic analog can compete with a corresponding known ligand for binding to a site on a receptor.

A candidate ligand can be contained within a population of characterized or uncharacterized molecules. As used herein, the term “population” refers to a group of two or more different molecules. A population can be as small as two molecules or as large as the number of individual molecules available to be assayed.

A population of ligands can be homogeneous to the extent that the population contains only one type of ligand, or heterogeneous to the extent that the population contains a variety of types of molecules. Exemplary homogenous populations of ligands include libraries of chemical compounds, such as combinatorial libraries, and populations of peptides or peptidomimetics. Exemplary heterogeneous populations of ligands include naturally-occurring populations of molecules, such as those contained in lysates prepared from cells, tissues, organs or organisms, as well as manmade mixtures of structurally unrelated potential drug molecules, such as mixtures of peptides, peptidomimetics, and small molecules. A candidate ligand can be produced using a variety of synthetic approaches, including combinatorial library synthesis methods. An exemplary approach for preparing a combinatorial library of organic molecules is described, for example, in Tan et al., J. Am. Chem. Soc. 121:9073-9087 (1999), incorporated herein by reference.

Binding of the compositions of the invention can be determined utilizing conventional detection systems known for detection of the specific detectable moiety present in the composition. Binding to a receptor of a composition comprising a common ligand can be assayed, for example, spectroscopically by detection of fluorescence, light absorption, light scattering, fluorescence polarization, chemiluminescence, and the like. Alternatively, composition binding can be assayed by detecting radioactivity emitted by a radioactive tag. Determination of the appropriate detection method for a given detectable moiety is within the level of skill of the ordinary artisan and can be determined through routine methods.

As the number of potential drug targets grow as a result of genome sequencing and genomics-based target discovery, the desire for simpler, more sensitive methods for detecting putative receptor molecules and more sensitive screening formats also increases. Simpler screening can be accomplished, for example, by minimizing the number of steps and reagents required for detection of ligand binding. More sensitive screening can be accomplished, for example, by the use of improved compositions that are detectable at low concentrations. The methods and compositions of the invention provide these advantages.

The methods of the invention include ligand binding assays that involve multiple washing and mixing steps, and assays that are “mix and measure,” and do not require multiple washing steps. Such assay formats are well known to those skilled in the art. A variety of homogeneous assays can be used in the detection methods of the invention, including Scintillation Proximity (SPA), Homogeneous Time-Resolved Fluorescence (HTRF), Lanthanide Chelation Excitation (LANCE), Fluorescence Polarization (FP), Fluorescence Correlation Spectroscopy (FCS), Fluorescence Resonance Energy Transfer (FRET), and Fluorescence Lifetime Measurement (FLM) assays.

SPA™ and HTRF/LANCE both rely on the biological interaction that occurs between a pair of molecules, for example, a receptor/ligand pair. During this interaction, the receptor and ligand are brought into close proximity on a molecular scale. In SPA, which is a radioactivity-based assay (Amersham, Bucking-hamshire, UK), the SPA bead contains a fluor that emits a photon of light upon excitation by a beta particle. In a typical assay format, the SPA bead is coated with a receptor, and the ligands to be tested are radioactively labeled. Upon a biologically relevant interaction, beta particles emitted by the radiolabeled ligand excite the fluor contained within the bead. The excited fluor then emits light of a given wavelength that can be captured by a detector and measured.

In comparison, the HTRF/LANCE assay format is based upon energy transfer that occurs when a donor-acceptor pair of molecules are brought close to each other. In this manner, a receptor-ligand interaction can be assayed by labeling the receptor with a donor molecule, through covalent attachment of the donor molecule to the receptor. Chelated Europium can be utilized for this purpose. The chelation prevents the Europium from being quenched by the environment. The acceptor moiety in this format is an allophycocyanin (e.g., XL-665) that accepts the energy from the Europium donor and emits the energy at a different wavelength. Europium has a long fluorescence lifetime, on the order of microseconds. Upon excitation, the Europium holds the energy for the length of its fluorescence lifetime, and then releases this energy, via energy transfer, to the acceptor molecule. This time lag is utilized in the assay to screen out contaminating signal from background that has a short fluorescence lifetime.

Fluorescence polarization provides another powerful method for homogeneous, solution-based non-radioactive assays. Fluorescence polarization is based on the principle that a small molecule, the fluorescent moiety, tumbles rapidly in solution, and if plane-polarized light is shone on such molecules, rapid tumbling during the lifetime of emission depolarizes the light. If, however, the fluorescent moiety is restricted in its tumbling via attachment to a large molecule, then during the fluorescence lifetime of the molecule, the polarity remains intact and the emission is polarized. This restriction of rotation and tumbling as a result of a biological interaction translates into a physically measurable quantity; the extent of incident light that remains polarized versus the fraction that gets depolarized as a result of free rotation of the molecules in solution. Fluorescence polarization readout therefore provides an average of the ensemble of molecules in solution. As such, fluorescence polarization provides the possibility of measuring the extent of binding in a given reaction in vitro. The polarization can be measured and the ratio of bound molecules to free molecules can be calculated.

For fluorescence polarization, the fluorescence lifetime of the fluor is in a range suitable for detecting fluorescence polarization. The fluorescence lifetime of the fluor is on the order of the correlation time for tumbling of the receptor and is generally in the range of 2 to 50 nanoseconds. Exemplary fluors having a fluorescence lifetime suitable for fluorescence polarization include fluorescein, BODIPY, Cy3, CY5 and Texas Red.

For example, when a high molecular weight fluorescent-common ligand-NAD binding receptor complex is excited with plane polarized light, the emitted light remains highly polarized because the molecular complex containing the fluorophore is constrained from rotating between the time the light is absorbed and emitted. When a low molecular weight fluorescent-common ligand is free in solution and excited by plane polarized light, it rotates faster than the corresponding bound complex, and the fluorescent-common ligands become randomly oriented during the time the light is absorbed and emitted, so that the emitted light is much less polarized. Examples of fluorescence polarization are provided in Sem et al., Biochemistry, 37:16069-16081 (1998).

As described in Example 6 below, addition of unlabeled candidate ligand to a fluorescent common ligand:NAD binding receptor complex will displace the fluorescent common ligand and the fluorescence polarization will decrease. Fluorescence polarization provides a quantitative means for measuring the amount of fluorescent common ligand:NAD binding receptor complex remaining in a competitive assay. From calculations based on a standard curve, the quantity of competing candidate ligand can be ascertained.

Any binding assay suitable for the purposes of the invention can be utilized. The assay can be conducted in solution phase or on a solid support and can be qualitative or quantitative. It can detect a single endpoint or can measure binding kinetics and can be conducted manually or can be automated. The methods of the present invention not only can be practiced with a single receptor, candidate ligand, and composition, but also with a plurality of receptors, candidate ligands and compositions, if desired.

In one embodiment, the present invention provides methods for the detection of putative receptors having NAD binding activity, for example, oxidoreductase receptors. These methods evaluate the ability of a putative receptor to bind compositions of the present invention. If binding occurs, the putative receptor has NAD binding activity and is a NAD binding receptor. Generally, these methods comprise contacting a putative receptor with a composition of the present invention and detecting the presence of a composition:receptor complex.

In another embodiment, the present invention provides methods for screening candidate ligands to determine their ability to bind a NAD binding receptor. These methods evaluate whether a particular compound can bind to a NAD binding site of a receptor and function as a NAD common ligand. These methods of the invention are based on competitive assays with a detectable composition of the invention. Thus, binding to a NAD binding site can be determined by competitive binding for NAD.

In one instance, the NAD binding receptor, composition of the invention, and candidate ligand can be contacted together and binding of the candidate ligand evaluated. In a second instance, a composition of the invention and a NAD binding receptor are bound to form a composition:NAD binding receptor complex. Then, the composition:NAD binding receptor complex and the candidate ligand are contacted. The ability of the candidate ligand to displace the composition from the composition:NAD binding receptor complex is then evaluated. In another instance, a candidate ligand and a NAD binding receptor are bound to form a candidate ligand:NAD binding receptor complex. Then, the candidate ligand:NAD binding receptor complex and a composition of the invention are contacted. The ability of the composition to displace the candidate ligand from the candidate ligand:NAD binding receptor complex is then evaluated.

Generally, for each of the methods of the invention, the time for incubation is at least about 5 minutes, more usually at least about 15 minutes, before exposing the mixture to a light source. Moderate, usually constant, temperatures are normally employed for the incubation. Incubation temperatures will normally range from about 5 to 99° C., for example from about 15 to 70° C., or 20 to 45° C. Temperatures during measurements will generally range from about 10 to 70° C., for example from about 20 to 45° C., for example, about 20 to 37° C., usually 20 to 25° C. After the appropriate incubation period, binding of the receptor to the candidate ligand is detected. One skilled in the art will readily recognize that variation of these exact parameters may be advantageous to the practice of the invention. Variation of these parameters to provide optimum conditions is within the level of skill of the ordinary artisan. Such variations are considered to be part of the present invention.

Detection methods of the invention will be described generally with regard to fluorescent detectable moieties. However, it is understood by those of ordinary skill in the art that the invention can be similarly practiced using other detectable moieties of the invention. Fluorescence-based technology is particularly useful as a sensitive system for assay detection. For example, U.S. Pat. No. 5,876,946 reports a high throughput assay for screening candidate compounds for inhibition of ligand-receptor interactions in which the inhibitor compound or the ligand is fluorescently labeled. For example, Estrogen Receptor CoreHTS™ Assay (Panvera Corp; Madison, Wis.) can be used, or assays such as those described in Sem et al., Biochemistry, 37:16069-16081 (1998) can be used.

Fluorescent detection systems are well known in the art. For example, fluorescent moieties can be illuminated at an appropriate excitation wavelength and detected fluorescence of the moieties can be detected at an appropriate emission wavelength. Furthermore, the fluorescent moieties can be illuminated with polarized light and the change in polarization during or after binding of the NAD cofactor to a receptor can be detected.

In one embodiment, the present invention provides methods for the detection of putative receptors having NAD binding activity. Several examples of this embodiment are provided below. However, it is understood that one of ordinary skill in the art would recognize routine variations which are encompassed by the present invention.

For example, the present invention provides a method for detecting binding activity of a putative NAD binding receptor, which comprises contacting the putative receptor with a composition comprising a detectable moiety linked to a common ligand and measuring binding of the common ligand to the receptor.

The invention provides a method for detecting binding activity of a putative NAD binding receptor which involves contacting the putative receptor with a composition of the invention for a time and under conditions sufficient for binding of the NAD cofactor to the receptor, optionally separating receptor bound compositions from unbound compositions, and measuring the presence of composition bound to the receptor. The presence of binding indicates that the putative receptor has NAD binding activity and is a NAD binding receptor as defined herein. Thus, the methods of the invention can be used to determine NAD binding activity of a newly identified gene.

The invention also provides a method for detecting binding activity of a putative NAD binding receptor which involves contacting the putative receptor with a composition of the invention, illuminating the composition:NAD binding receptor complex with a light source, and detecting the emission of the complex. The change in emission intensity is indicative of NAD binding activity. Emission intensity can increase or decrease upon binding of a receptor and is dependent upon whether the presence of the receptor results in more or less fluorescence quenching relative to solvent. Evaluation of the level of emission further can be used to quantitate the binding affinity of the NAD binding receptor. In addition to measuring emission intensity, one can measure the degree of polarization of the emitted light if the excitation is with polarized light. The degree of polarization will be a function of the extent of binding of the composition to the receptor.

For detection of putative receptors having NAD binding activity, the invention can be practiced employing any of the common ligands, including oxidoreductase common ligands, such as NAD common ligands and dye common ligands disclosed herein. Similarly, the methods can be practiced employing any of the detectable moieties of the present invention and using any of the detection methods disclosed herein.

In another embodiment, the present invention provides methods for screening candidate ligands for binding to a NAD binding receptor. These methods of the invention are competitive assays. Several examples of this embodiment are provided below. However, it is understood that one of ordinary skill in the art would recognize routine variations which are encompassed by the present invention.

For example, the invention provides a method for screening candidate ligands for binding to a NAD binding receptor which involves contacting a NAD binding receptor, a composition of the invention, and a candidate ligand, and measuring binding of the candidate ligand to the NAD binding receptor.

The present invention also provides a method for screening candidate ligands for binding to a NAD binding receptor, which comprises contacting a NAD binding receptor with a candidate ligand and a composition comprising a common ligand linked to a detectable moiety; and measuring binding of the candidate ligand to the NAD binding receptor.

The invention also provides a method for screening candidate ligands for binding to a NAD binding receptor which involves contacting a NAD binding receptor and a composition of the invention to form a composition:NAD binding receptor complex, contacting the composition:NAD binding receptor complex with a candidate ligand, and and measuring displacement of the composition by the candidate ligand.

The invention further provides a method for screening candidate ligands for binding to a NAD binding receptor which involves contacting a NAD binding receptor and a candidate ligand to form a candidate ligand:NAD binding receptor complex, contacting the candidate ligand:NAD binding receptor complex with a composition of the invention, and measuring displacement of the candidate ligand by the composition.

For screening of candidate ligands for NAD binding activity, the invention can be practiced employing any of the common ligands, including oxidoreductase common ligands, NAD common ligands, or dye common ligands disclosed herein. Similarly, the methods can be practiced employing any of the detectable moieties of the present invention. Further, the invention can be practiced employing any of the NAD binding receptors, either known or newly identified by the methods disclosed herein, and any of the detection methods herein.

In a further embodiment, candidate ligands which bind to the NAD binding receptor can be screened for modulation of receptor activity. The modulation of NAD binding receptor activity can constitute inhibition or potentiation of receptor activity.

The invention further provides a method for detecting the binding activity of a putative receptor, such as a putative oxidoreductase, employing HitHunter™ EFC technology. This method utilizes a genetically engineered β-galactosidase (β-gal) enzyme that contains two fragments. The fragments are termed Enzyme Acceptor(EA) and Enzyme Donor (ED). When the two fragments are separated, the enzyme is inactive. When the fragments are together they can recombine spontaneously to form active enzyme by a process called complementation. See, for example, U.S. Pat. No. 5,434,052, incorporated herein by reference.

In this embodiment, the present invention utilizes an ED-common ligand conjugate. When the common ligand binds to the putative receptor, formation of the ED-EA complex is inhibited and, thus, the β-gal enzyme is inactive. When the common ligand does not bind to the putative receptor, the ED is free to form a complex with the EA and active β-gal enzyme can be detected.

This method also can be employed as a quantitative assay to measure the amount of a candidate ligand that binds to a NAD binding receptor. The quantitative assay measures ED-common ligand conjugate in the presence of EA and compares measurements in the presence of candidate ligand with measurements in the absence of candidate ligand. The method can be employed with any of the compositions of the invention, including those comprising a NAD common ligand linked to a detectable moiety, those comprising a dye common ligand linked to a detectable moiety, and those comprising a dye common ligand.

The methods of the invention can be employed to simultaneously identify a plurality of candidate ligands that can bind to a NAD binding receptor. Such methods involve obtaining a population of candidate ligands, a plurality of NAD binding receptors, and compositions of the invention, contacting a population of candidate ligands with the plurality of NAD binding receptors for a time and under conditions sufficient for binding, exposing the plurality of NAD binding receptors to a composition of the invention, and detecting binding of candidate ligands to NAD binding receptors.

In one embodiment, the method of the invention can conveniently be used to assay a plurality of samples that can be detected by fluorescence polarization. The invention provides a method of detecting fluorescence which includes the steps of illuminating a sample containing a NAD binding receptor, a fluorescent common ligand and a candidate ligand with polarized light; and measuring the fluorescence polarization of the sample. A decrease in fluorescence polarization in the presence of the candidate ligand indicates that the candidate ligand competes with the fluorescent common ligand and therefore binds to a NAD binding site of the receptor. For example, a sample containing LDH, fluorescent NAD and NADH are illuminated with polarized light and the resulting fluorescence polarization is measured.

In a further embodiment of the invention, the methods involve contacting a population of putative receptors with a plurality of candidate ligands that can bind to a NAD binding receptor and compositions of the invention under conditions sufficient for binding. As used herein, conditions sufficient for binding will vary depending upon the characteristics of the receptors and ligands and the types of samples in which the receptors and ligands are contained. Receptors and ligands can be in solution, or either can be attached to a solid support, such as a bead, assay plate or other surface. Conditions that allow interactions between macromolecular receptors and small molecule candidate ligands are well known to those skilled in the art.

In a further embodiment, the invention is directed to the screening of candidate ligands that can bind to a NAD binding receptor without the necessity for washing and separation steps. These assays advantageously require fewer manipulative steps than traditional assays, which require washing steps. As such, these assays typically are faster, have lower error and are particularly well-suited for automation. In another embodiment, the invention is directed to the identification of candidate ligands that bind to a NAD binding receptor in a sandwich assay format. In the sandwich assay format a NAD binding receptor can be immobilized, for example, bound to a microtiter plate, although immobilization is not required.

In accordance with another embodiment of the present invention, there are provided detection systems, in kit form, comprising at least one composition of the invention in a suitable packaging material. In one embodiment, for example, the detection system of the invention includes NAD, NADH, NADP or NADPH coupled to a detectable moiety. For example, the detection system can comprise a reduced or oxidized NAD coupled to a fluorescent moiety. Alternatively, the detection system can comprise a NAD cofactor coupled at the N⁶ position of the adenine ring to a fluorescein derivative.

In another embodiment, for example, the detection system of the invention includes a dye common ligand coupled to a detectable moiety. For example, the detection system can comprise Reactive Red 120, Reactive Green 5, or Reactive Blue 2 coupled to a fluorescent moiety. In yet another embodiment, the detection system of the invention includes a dye common ligand such as Reactive Red 120, Reactive Green 5, or Reactive Blue 2.

The detection system can further comprise a NAD binding receptor as described herein. In one embodiment, for example, the detection system includes an oxidoreductase. For example, the detection system can comprise dihydrodipicolinate reductase, enoyl ACP reductase, alcohol dehydrogenase, lactate dehydrogenase, or glyceraldehyde-3-phosphate dehydrogenase. The detection system can further contain instructions for practicing the methods of the invention. The detection system is useful for the determination of putative receptors and, when a NAD binding receptor is included, for detection of ligands that bind to an oxidoreductase.

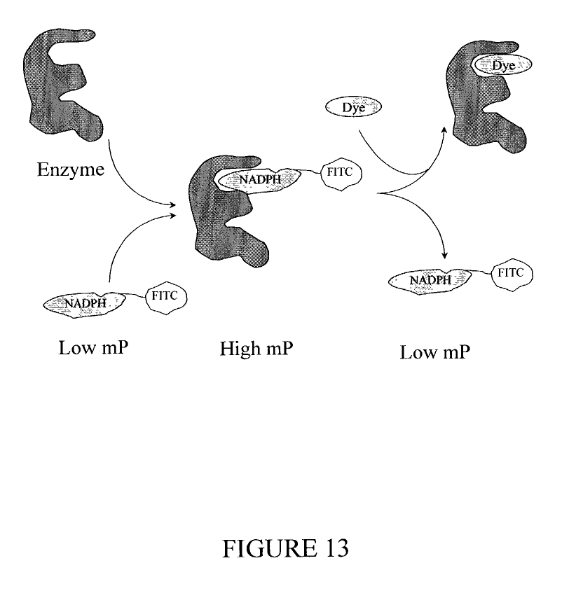

The competitive binding of the dye common ligands of the present invention can be evaluated by use of a displacement assay. A schematic of such a displacement assay is provided in FIG. 13. In particular, enzyme in the presence of a competitive ligand will have less FITC-NADPH bound if both ligands compete for the same site. When the displacing ligand is present at a sufficiently high concentration, FITC-NADPH will be completely displaced. The dye common ligands of the present invention can completely displace FITC-NADPH from complexes containing this fluorescent tracer and a dehydrogenase. This displacement indicates that the ligand binds to the NAD binding site of the enzyme. Thus, dye common ligands of the invention are useful in displacement assays to study the activity of an oxidoreductase, particularly those having unknown function.

The invention is directed to compositions that bind to the NAD binding site of a NAD binding receptor. In some cases, a common ligand of the invention will bind to essentially all members of a family of NAD binding receptors, such as oxidoreductases. For example, a NAD common ligand such as NAD will bind to a substantial portion of all oxidoreductases. However, it is understood that a common ligand of the invention need not bind to all NAD binding receptors so long as the common ligand binds to at least two NAD binding receptors. For example, a common ligand can bind to a subfamily of a NAD binding receptor, such as oxidoreductases, for example, a pharmacophore family that binds NAD, NADH, NADP, or NADPH in a particular conformation (see U.S. application Ser. No. 09/747,174, which is incorporated herein by reference).

The identification of a common ligand of the invention that binds to a subset of NAD binding receptors can be useful as a common ligand for a subfamily of receptors. Such a common ligand can be used to identify a member of a subfamily, which allows the use of targeted libraries for the particular subfamily. Targeted libraries can be focused to optimize binding to a receptor subfamily that have more similar binding properties than the receptor family as a whole. The use of libraries targeted to a particular subfamily allows more efficient screening and identification of compounds that specifically bind to the receptor in the corresponding subfamily; see, for example, copending U.S. application Ser. No. 10/032,395, filed Dec. 21, 2001, incorporated by reference herein.

In performing the methods of the invention, one skilled in the art can readily determine appropriate conditions and controls useful for a particular application. For example, when determining the binding of a common ligand of the invention to a NAD binding receptor, a control can be the detectable common ligand in the absence of the receptor. When a candidate ligand is being tested for binding, a control can be the receptor and a detectable common ligand in the absence of the candidate ligand. One skilled in the art will readily recognize these and other suitable conditions and controls for detecting desired binding activity as described herein.

The following examples are intended to illustrate but not limit the present invention.

### EXAMPLE 1

### Preparation of Fluorescent NADH and Fluorescent NADPH

This example demonstrates the synthesis of fluorescent NADH and fluorescent NADPH employing the reaction scheme depicted in FIG. 1. N⁶-(2-Aminoethyl)-NAD was prepared according to the literature (Andreas F. Buckman and Victor Wray, Biotechnology and Applied Biochemistry 15:303-310 (1992)). The N⁶-(2-Aminoethyl)-NAD was characterized using Nuclear Magnetic Resonance (NMR)and Mass Spectrometry (MS) analysis. The structure was confirmed by ¹H and ³¹P NMR: ¹H NMR (D₂O, pH 7) 9.39 (s, 1 H), 9.24 (d, J=6.4 Hz, 1 H), 8.89 (d, J=8.0 Hz, 1 H), 8.41 (s, 1 H), 8.23 (m, 2 H), 6.10 (d, J=5.5 Hz, 1 H), 6.03 (d, J=5.5 Hz, 1 H), 4.50 (m, 4 H), 4.41 (m, 1 H), 4.36 (m, 2 H), 4.22 (m, 3 H), 3.64 (m, 3 H), 3.33 (t, J=5.4 Hz, 3 H) ppm; ³¹P NMR (D₂O, H₃PO₄) −10.9 (dd, J=50, 80 Hz, 2 P) ppm; MS (ESI), m/z (relative intensity) 706 (M+1⁺, 100).

N⁶-(2-Aminoethyl)-NADP was prepared according to the literature (Andreas F. Buckmann, European Patent Application 0247537 (1987); Andreas F. Buckmann, Biocatalysis 1:173-186 (1987)). N⁶-(2-Aminoethyl)-NADP was purified by reversed-phase High Performance Liquid Chromatography (HPLC) using a preparative Supelcosil™ ABZ column, Supelco, acetonitrile/water with 0.025% of trifluoacetic acid, 0-5 minutes 100% of water and 5-14 minutes 0-10% of acetonitrile. The N⁶-(2-Aminoethyl)-NADP was characterized using NMR and MS analysis. The structure was confirmed by ¹H and ³¹P NMR: ¹H NMR (D₂O, pH 7) 9.45 (s, 1 H), 9.31 (d, J=6.2 Hz, 1 H), 8.99 (d, J=8.0 Hz, 1 H), 8.63 (s, 1 H), 8.47 (s, 1 H), 8.33 (t, J=6.5 Hz, 1 H), 6.33 (d, J=5.3 Hz, 1 H), 6.21 (d, J=5.3 Hz, 1 H), 5.11 (m, 1 H), 4.61 (m, 4 H), 4.50 (m, 4 H), 4.28 (m, 4 H), 3.42 (t, J=5.8 Hz, 3 H) ppm; ³¹P NMR (D₂O, H₃PO₄) 0.13 (s, 1 P), −11.1 (dd, J=49, 71 Hz, 2 P) ppm; MS (ESI), m/z (relative intensity) 787 (M+1⁺, 100).

For the preparation of fluorescent NAD, fluorescein isothiacynate (79 mg, 0.20 mmol) was added to a solution of N⁶-(2-aminoethyl)-NAD (108 mg, 0.15 mmol) and Na₂CO₃ (71 mg, 0.67 mmol) in 6 mL of H₂O and THF (1:1). After stirring for 3 hours, the pH was adjusted to 6.0 with 0.5 M HCl. Purification by reversed-phase High Performance Liquid Chromatography (HPLC) (preparative C₁₈ column, acetonitrile/water with 0.025% of trifluoacetic acid, 0-20 minutes 10-90% acetonitrile and collecting the peak at 6.7 minutes) gave 136 mg (79.5%) of fluorescent NAD. The fluorescent NAD was characterized using NMR and MS analysis. The structure was confirmed by ¹H and ³¹P NMR: ¹H NMR (D₂O, pH 7) 9.27 (s, 1 H), 9.10 (d, J=5.1 Hz, 1 H), 8.69 (s, 1 H), 8.23 (s, 1 H), 8.07 (s, 1 H), 7.99 (s, 1 H), 7.65 (s, 1 H), 7.30 (s, br, 1 H), 6.88 (m, 3 H), 6.55 (m, 5 H), 6.00 (m, 1 H), 5.85 (m, 1 H), 4.60-4.0 (m, 11 H), 3.89-3.50 (m, 4 H) ppm; ³¹P NMR (D₂O, H₃PO₄) −11.0 (dd, J=52, 84 Hz, 2 P) ppm; MS (ESI), m/z (relative intensity) 1096 (M+1⁺, 100).

The same procedure was used for the preparation of fluorescent NADP (129 mg) from N⁶-(2-aminoethyl)-NADP (100 mg, 0.11 mmol) and fluorescein isothiacynate (90 mg, 0.23 mmol) in 68.1% yield. The peak at 5.5 minutes was collected. The fluorescent NADP was characterized using NMR and MS analysis. The structure was confirmed by ¹H and ³¹P NMR: ¹H NMR (D₂O, pH 7): 9.33 (s, 1 H), 9.20 (d, J=5.5 Hz, 1 H), 8.81 (d, J=7.5 Hz, 1 H), 8.41 (s, 1 H), 8.21 (m, 2 H), 7.82 (s, 1 H), 7.53 (m, 1 H), 7.16 (m, 3 H), 6.75 (m, 5 H), 6.07 (m, 2 H), 4.84 (m, 1 H), 4.55 (m, 4 H), 4.40-4.10 (m, 7 H), 4.05-3.70 (m, 4 H) ppm; ³¹P NMR (D₂O, H₃PO₄) 0.27 (s, 1 P), −11.0 (dd, J=51, 83 Hz, 2 P); MS (ESI), m/z (relative intensity) 1176 (M+1⁺, 100).

For the preparation of fluorescent NADH from fluorescent NAD, sodium hydrogensulfite (38 mg, 0.18 mmol) was added to a solution of fluorescent NAD (33 mg, 0.03 mmol) in 2 mL of 3% NaHCO₃ over a period of 1 h. The reaction was continued for another 1 h. Oxygen was passed through the solution for 0.5 h. Reversed-phase High Performance Liquid Chromatography (HPLC) purification by collecting the peak at 7.6 minutes using a preparative C₁₈ column, acetonitrile/water with 0.025% of AcONH₄, 0-15 minutes 10-80% acetonitrile afforded 32 mg (97.0%) of fluorescent NAD. Signals in the proton spectrum were complex due to the presence of more than two isomers. The structure was confirmed by ³¹P NMR and MS analysis: ³¹P NMR (D₂O, H₃PO₄) −10.7 (dd, J=47, 96 Hz, 2 P); MS (ESI), m/z (relative intensity) 1096 (M−1⁺, 100).

Fluorescent NADPH (16 mg) was prepared from fluorescent NADP (28 mg, 0.023 mmol) in 57.1% yield, using the procedure described above. The pure fluorescent NADPH was obtained by collecting the peak at 10 minutes using a preparative Supelcosil™ ABZ column, acetonitrile/water with 0.025% of AcONH₄, 0-3 minutes 10% of acetonitrile, 3-15 minutes 10-90% of acetonitrile. Signals in the proton spectrum were complex due to the presence of more than two isomers. The structure was confirmed by ³¹P NMR and MS analysis: ³¹P NMR (D₂O, H₃PO₄) 1.28 (s, br, 1 P), −10.4 (m, br, 2 P); MS (ESI), m/z (relative intensity) 1176 (M−1⁺, 100).

### EXAMPLE 2

### Determination of the Concentration of FITC-NADH for Fluorescence Polarization Displacement Assays

This example illustrates the determination of the concentration of FITC-NADH that would provide the best signal to noise ratio for fluorescence polarization assay. FITC-NADH was dissolved in 10 mM TAPS buffer with 5% NaHCO₃, pH=8. The final concentration of FITC-NADH was 150 μM. In later experiments 150 μM FITC-NADH was diluted to the desired concentration in 20 mM Potassium Phosphate buffer, pH=7.4. Fluorescence polarization was monitored using a Beacon 2000 Variable Temperature Fluorescence Polarization System (PanVera Corporation, Madison, Wis.) and an LJL Analyst HT 96-384 (Molecular Devices Corporation, Sunnyvale, Calif.). The concentration of FITC-NADH as fluorescent tracer was selected by measuring fluorescence polarization of varying concentrations of FITC-NADH in 10 mM Potassium Phosphate buffer, pH=7.4. The concentration range was 0.15 nM—15 nM. For the best signal to noise ratio regarding both the horizontal and vertical intensities that contribute to the fluorescence polarization value, a concentration of 25 nM was chosen for FITC-NADH in the majority of experiments.

The data in Table 1 clearly indicate that 0.5 nM is the minimum FITC-NADPH concentration that can be employed.

TABLE 1

| FITC-NADH, nM | mP |
|---|---|
| 15 | 45.8 |
| 5 | 44.4, 55.1, 49.4, 47 |
| 1.5 | 43.6, 42.4, 43.7, 41.8 |
| 0.5 | 48.5 |
| 0.15 | 21.6, 31, 23.9, 26.8, 34.6 |

### EXAMPLE 3

### Binding of FITC-NADH to Dehydrogenases

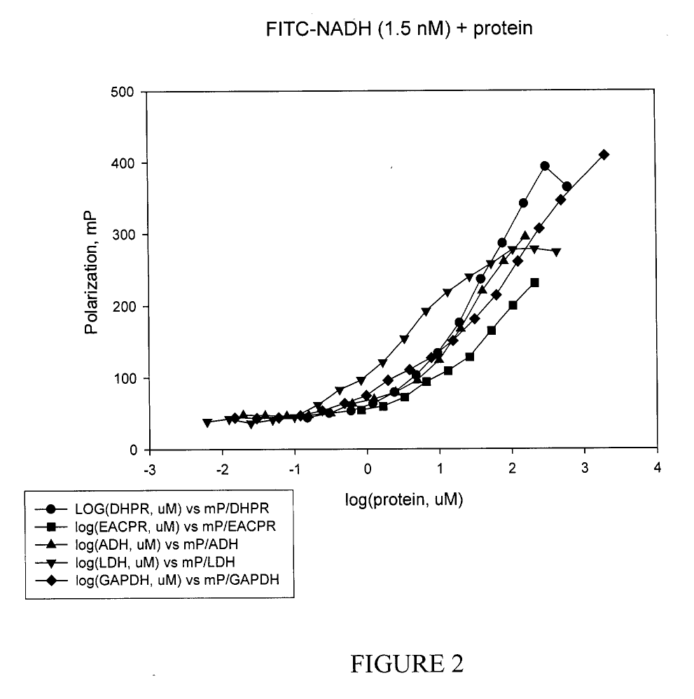

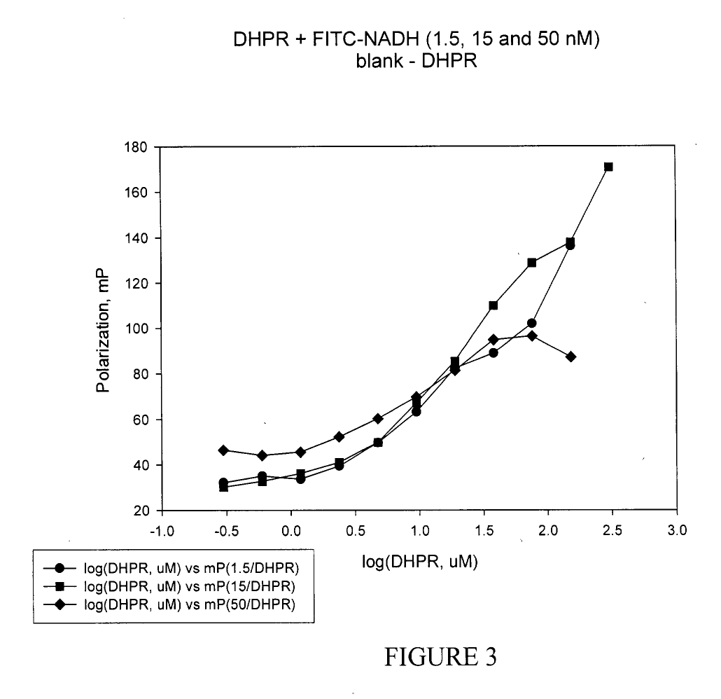

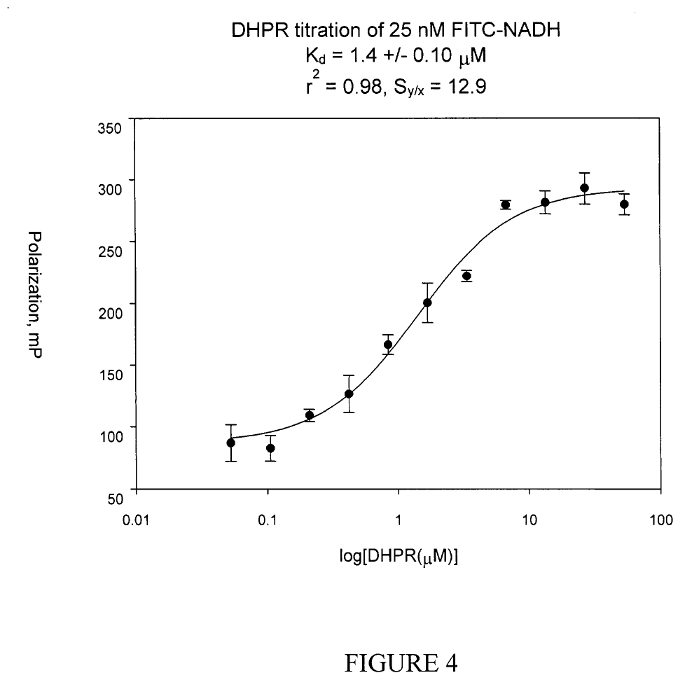

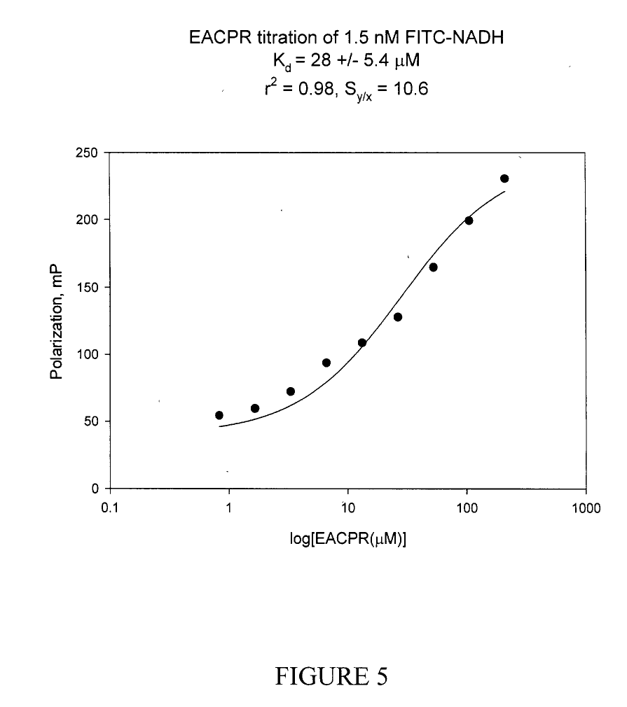

This example illustrates the binding characteristics of FITC-NADH to various dehydrogenases. Protein was concentrated to the highest possible concentration using a Centricon YM-30 membrane, Millipore, then washed twice with 20 mM Potassium Phosphate buffer, pH=7.4, or 20 mM HEPES, pH 7.8. A series of two-fold dilutions of protein in 20 mM Potassium Phosphate buffer, pH=7.4, or 20 mM HEPES, pH 7.8 was prepared. An individual blank of protein in buffer was measured for each point. 150 μM FITC-NADH was diluted to the desired concentration in 20 mM Potassium Phosphate buffer, pH=7.4, or 20 mM HEPES, pH 7.8. FITC-NADH was added to the test tubes and the fluorescence polarization measurement was taken. The Log(protein, μM) versus Polarization (mP) (FIG. 2) represents the binding curves of FITC-NADH to the dehydrogenases dihydrodipicolinate reductase, enoyl ACP reductase, alcohol dehydrogenase, lactate dehydrogenase and glyceraldehyde-3-phosphate dehydrogenase at a concentration of 1.5 nM FITC-NADH. The plot of Log(DHPR) versus Polarization (mP) (FIG. 3) represents the binding curves of FITC-NADH to the dehydrogenase dihydrodipicolinate reductase at concentrations of 1.5, 15 and 50 nM FITC-NADH. In FIG. 4, the plot of Log(DHPR) versus Polarization (mP) represents the binding curve of FITC-NADH to the dehydrogenase dihydrodipicolinate reductase at 25 nM FITC-NADH. In FIG. 5, the plot of Log(EACPR) versus Polarization (mP) represents the binding curve of FITC-NADH to the dehydrogenase enoyl ACP reductase at 25 nM FITC-NADH.

To determine the K_{d} of FITC-NADH to the dehydrogenases the Log(DH) versus Polarization (mP) data were fitted to the following equation:

mP=YL+(YH−YL)*(1/(1+K_{d}/A))

where A is enzyme concentration, YH is the high polarization value for bound FITC-NADH, and YL is the low polarization value for unbound FITC-NADH.

From this equation the desired concentration of oxidoreductase was selected for future experiments to provide an initial polarization (mP) value of at least 45 units, for example, between 150 and 200 units.

### EXAMPLE 4

### Stability of Dehydrogenases and FITC-NADH

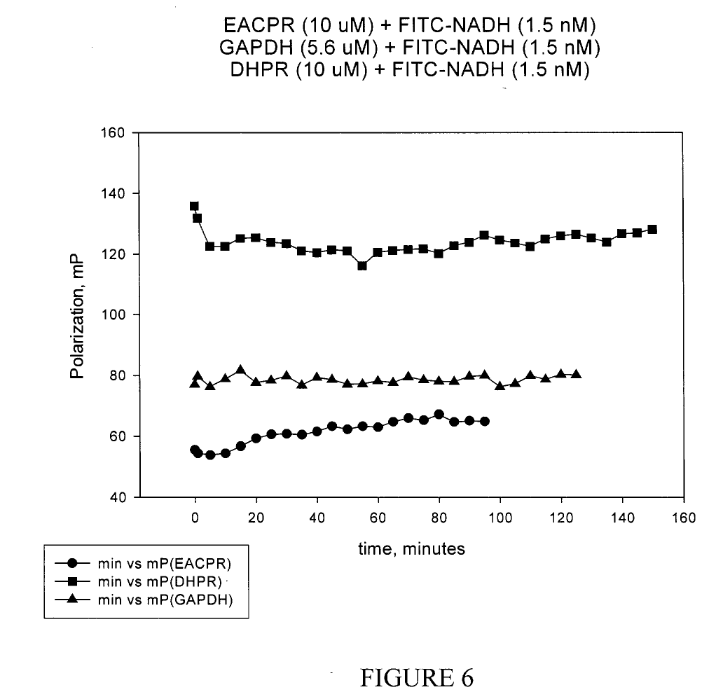

The fluorescence polarization of solutions of 10 μM enoyl ACP reductase, 5.6 μM glyceraldehyde-3-phosphate dehydrogenase and 10 μM dihydrodipicolinate reductase with 1.5 nM FITC-NADH, was measured. The concentration of the dehydrogenase was such that the solution of dehydrogenase and FITC-NADH would give the polarization mP value of 70-80. The blank was a solution of protein in buffer. Fluorescence polarization measurements were taken for 2 hours at 5 minute intervals. FIG. 6 shows stability curves of the dehydrogenases enoyl ACP reductase, dihydrodipicolinate reductase and glyceraldehyde-3-phosphate dehydrogenase with FITC-NADH plotted as time in minutes versus Polarization, mP. The data demonstrate that both enzyme and FITC-NADH are stable for 100 minutes, which is a desirable property of the screening reagent.

### EXAMPLE 5

### Displacement of FITC-NADH with NADH

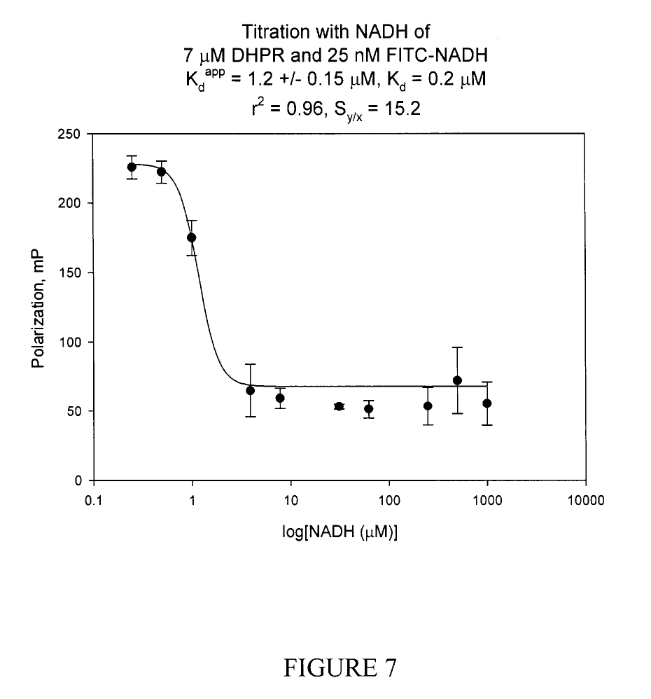

This example demonstrates the change in fluorescence polarization when NADH displaces FITC-NADH bound to a dehydrogenase. The fluorescence polarization of solutions of 7 μM dihydrodipicolinate reductase and 25 nM FITC-NADH with NADH was measured. The concentration of dihydrodipicolinate reductase was such that a solution of dihydrodipicolinate reductase and FITC-NADH would give the fluorescence polarization mP value of 220-230 mP. The blank was protein in buffer and NADH. FIG. 7 shows the results of the displacement assay plotted as Log(NADH) versus polarization, mP, for dihydrodipicolinate reductase. The Kapp was determined to be 1.2±0.15 μM. After correction for enzyme concentration, an actual K_{d} of 0.2 μM was obtained. The data was fitted to the following equation:

mP=YH′−(YH′−YL)*(1/(1+K_{d}^{app}/C))

K_{d}=K_{d}^{app}*(1/(1+A/K_{d}^{tracer}))

where C is the concentration of NADH; A is the concentration of enzyme; YH′ is the polarization for the FITC-NADH at the specified enzyme concentration in the absence of NADH; YL is the polarization for FITC-NADH when fully displaced from the enzyme by NADH; and K_{d}^{tracer} is the dissociation constant of the enzyme for FITC-NADH.

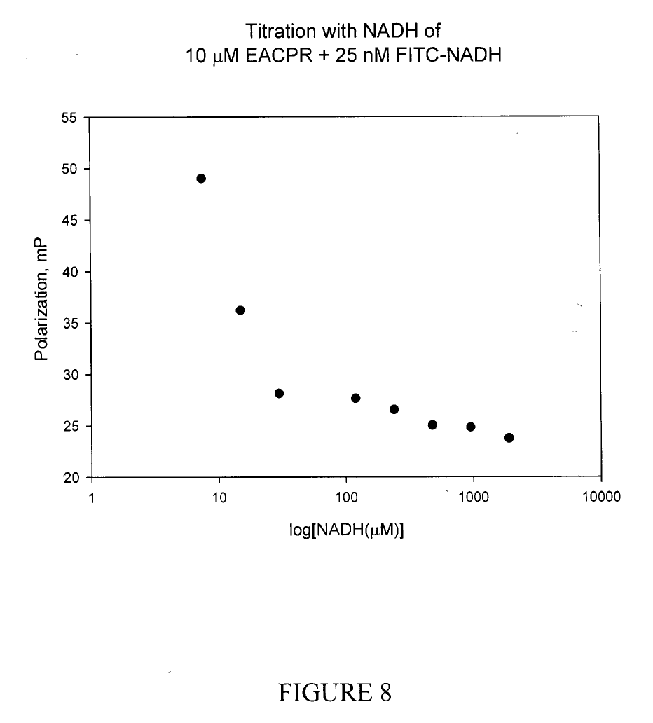

The fluorescence polarization of solutions of 10 μM enoyl ACP reductase and 25 nM FITC-NADH was measured in a similar manner. The concentration of enoyl ACP reductase was such that a solution of enoyl ACP reductase and FITC-NADH would give the fluorescence polarization, mP, value of about 48 mP. FIG. 8 shows the corresponding curve for enoyl ACP reductase titrated with NADH.

### EXAMPLE 6

### Displacement of FITC-NADH with an Inhibitor

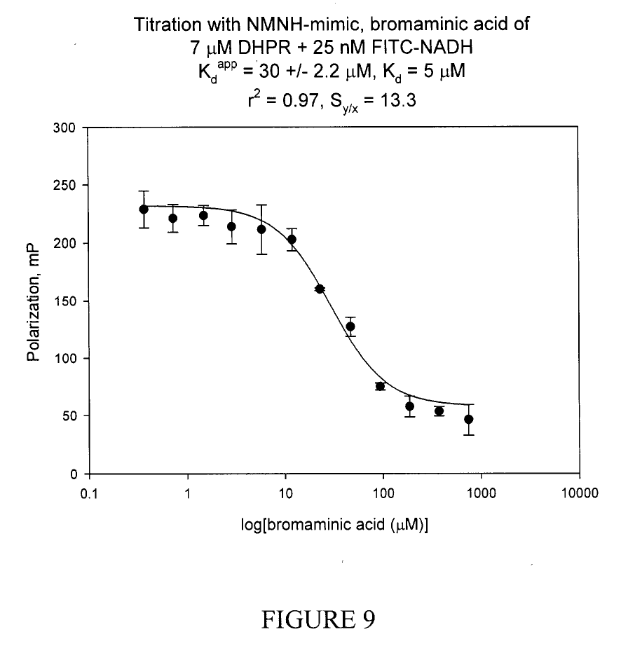

This example demonstrates the change in fluorescence polarization when an inhibitor displaces FITC-NADH bound to a dehydrogenase. The displacement of FITC-NADH from dihydrodipicolinate reductase in the presence of the candidate inhibitor compound bromaminic acid, an NMNH-mimic (nicotinamide mononucleotide mimic), was measured by fluorescence polarization assay. The concentration of dihydrodipicolinate reductase was such that dihydrodipicolinate reductase in the presence of FITC-NADH would give the fluorescence polarization, mP, value of 220-230. Protein in buffer combined with the inhibitor was used as the blank. The results of the displacement assay plotted as Log(bromaminic acid, μM) versus Polarization, mP, are shown in FIG. 9.

The Log(bromaminic acid, μM) versus Polarization (mP) data were fitted to the following equation:

mP=YH′−(YH′−YL)*(1/(1+K_{d}^{app}/C))

K_{d}=K_{d}^{app}*(1/(1+A/K_{d}^{tracer}))

where C is the concentration of NADH; A is the concentration of enzyme; YH′ is the polarization for the FITC-NADH at the specified enzyme concentration in the absence of NADH; YL is the polarization for FITC-NADH when fully displaced from the enzyme by NADH; and K_{d}^{tracer} is the dissociation constant of the enzyme for FITC-NADH. The K_{d}^{app} of inhibitor bromaminic acid to dihydrodipicolinate reductase was determined to be 30±2.2 μM. After correction for enzyme concentration, an actual Kd of 5 μM was obtained.

### EXAMPLE 7

### Identification of the Potent and Cross-reactive Ligands of Dehydrogenases Binding in the Active Site and their Application for Displacement Assay

Historically, various triazinyl dyes immobilized on insoluble resins were used for purification of oxidoreductases. They bind in the nucleotide-binding site of those enzymes, providing specificity to the binding. Targets that represent different subfamilies of the class of the dehydrogenases were screened against a set of the triazinyl dyes. Several of the dyes bound to a majority of the assayed dehydrogenases. These dyes can be used as probes for the detection of other ligands binding in the nucleotide site of the dehydrogenases.

In search for specific ligands with potent binding to several dehydrogenases we screened our internal enzyme panel. In brief, activity of the enzymes was measured in the presence and absence of the varying concentration of the dyes. The summary of the screening is provided in the following table (Table 2), with structures shown in FIG. 15.

TABLE 2

| Dehydrogenase | Dyes assayed |
|---|---|
| Dihydrodipicolinate reductase | 1. Reactive Green 5 |
| (DHPR) | 2. Reactive Green 19 |
| | 3. Reactive Orange 14 |
| | 4. Reactive Brown 10 |
| | 5. Reactive Yellow 86 |
| | 6. Naphthol Yellow S |
| | 7. Reactive Blue 2 |
| | 8. Reactive Red 120 |
| Deoxy-D-xylulose 5-phosphate | 1. Reactive Green 5 |
| reductoisomerase (DOXPR) | 2. Reactive Green 19 |
| | 3. Reactive Orange 14 |
| | 4. Reactive Brown 10 |
| | 5. Reactive Yellow 86 |
| | 6. Naphthol Yellow S |
| | 7. Reactive Blue 2 |
| | 8. Reactive Red 120 |
| Lactate dehydrogenase (LDH) | 1. Reactive Green 5 |
| | 2. Reactive Blue 2 |
| | 3. Reactive Red 120 |
| 3-Hydro-3-methyl-glutaryl-CoA | 1. Reactive Green 5 |
| reductase (HMGCoAR) | 2. Reactive Blue 2 |
| | 3. Reactive Red 120 |
| | 4. Reactive Blue 4 |
| | 5. Reactive Orange 14 |
| Enoyl-acyl carrier protein | 1. Reactive Green 5 |
| reductase (EACPR) | 2. Reactive Blue 2 |
| | 3. Reactive Red 120 |
| | 4. Reactive Blue 4 |
| | 5. Reactive Orange 14 |
| Shikimate dehydrogenase | 1. Reactive Green 5 |
| | 2. Reactive Blue 2 |
| | 3. Reactive Red 120 |
| Aspartate semialdehyde | 1. Reactive Green 5 |
| dehydrogenase | 2. Reactive Green 19 |
| | 3. Reactive Orange 14 |
| | 4. Reactive Brown 10 |
| | 5. Reactive Yellow 86 |
| | 6. Naphthol Yellow S |
| | 7. Reactive Blue 2 |
| | 8. Reactive Red 120 |

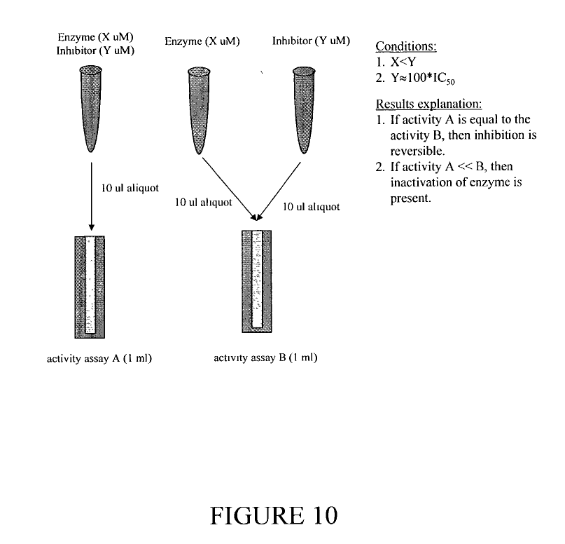

Three of the triazinyl dyes were found to inhibit most of the dehydrogenases (see Table 3). Since all of the dye molecules have a triazinyl reactive group, reversibility of the inhibition was checked. Schematics are presented in FIG. 10. Binding of those dyes was reversible, and no additional inactivation was observed.

TABLE 3

| | |
|---|---|
| Triazine | Inhibition (IC₅₀, nM) |

| Dye | DHPR | DOXPR | LDH | HMGCoAR | EACPR |
|---|---|---|---|---|---|
| Reactive | 230 | 270 | 590 | 44 | 210 |
| Green 5 | | | | | |
| Reactive | 220 | 230 | 9.7 | 360 | 246 |
| Red 120 | | | | | |
| Reactive | 1100 | 2000 | 1500 | 1000 | 2500 |
| Blue 2 | | | | | |

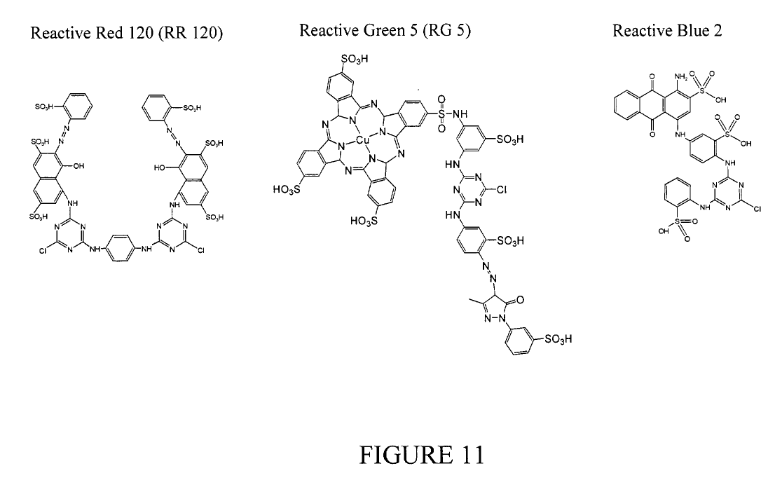

The values of the dissociation constants of the enzyme-dye complexes are expected to be lower than the corresponding values in Table 3 due to the competition with non-zero concentrations of the nucleotide substrate. The structures of these three dyes are provided in FIG. 11.

All of the compounds tested displayed full inhibition of assayed enzymes. FIG. 12 shows inhibition of one exemplified dehydrogenase, DOXPR, by Reactive Red 120 (RR120) and by Reactive Green 5 (RG5). In this assay, the oxidoreductase common ligand (NADPH) was employed at a concentration sixteen times its Kₘ value. Use of such a high concentration of oxidoreductase common ligand results in a calculated IC₅₀ value at least 17-fold that of the dissociation constant (K_{d}). The observed mode of inhibition is competitive versus oxidoreductase common ligand. Taking this into consideration, the estimated K_{d} value was calculated to be 13 to 16 nM.

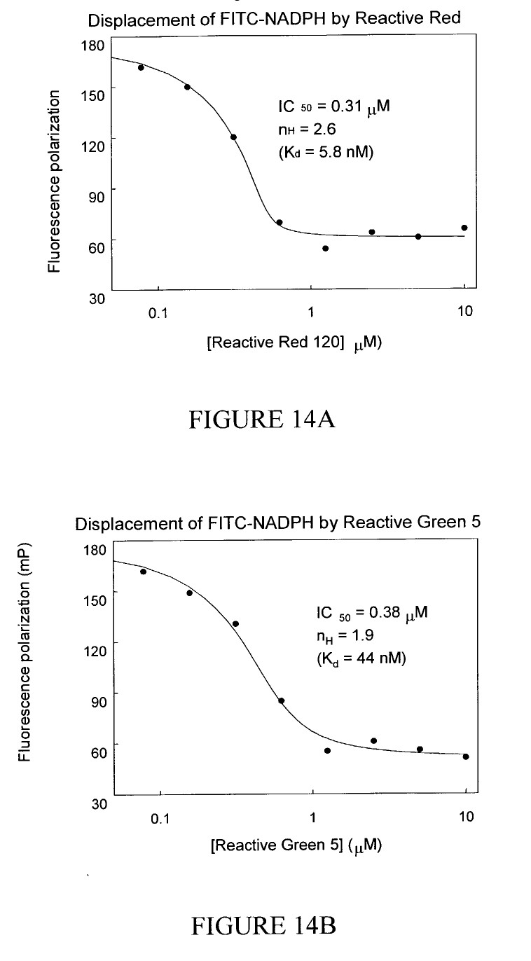

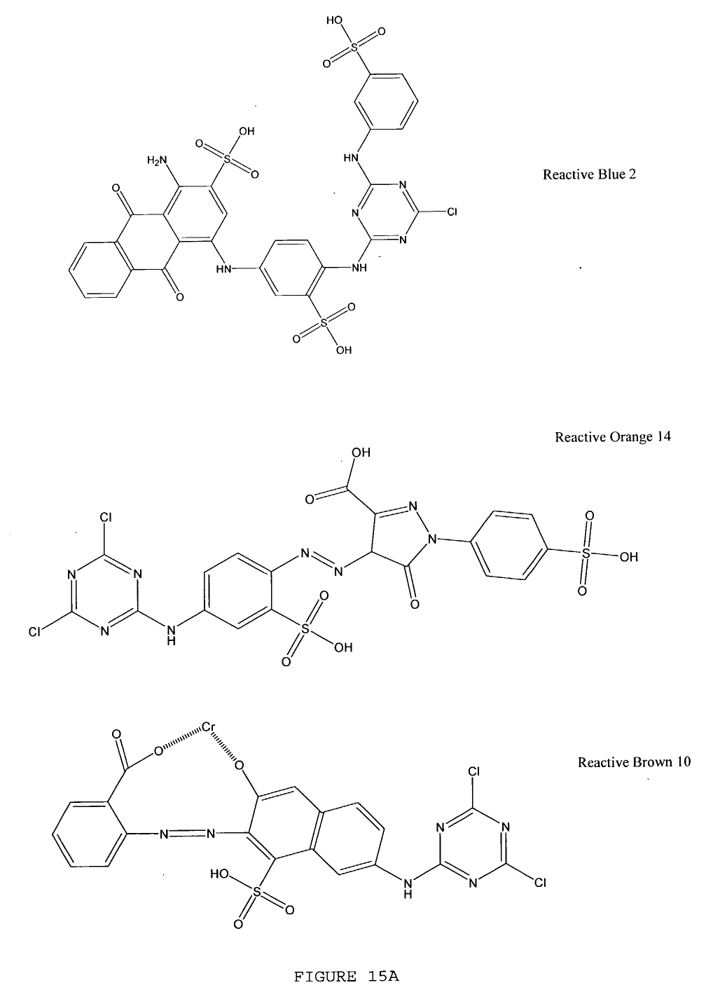

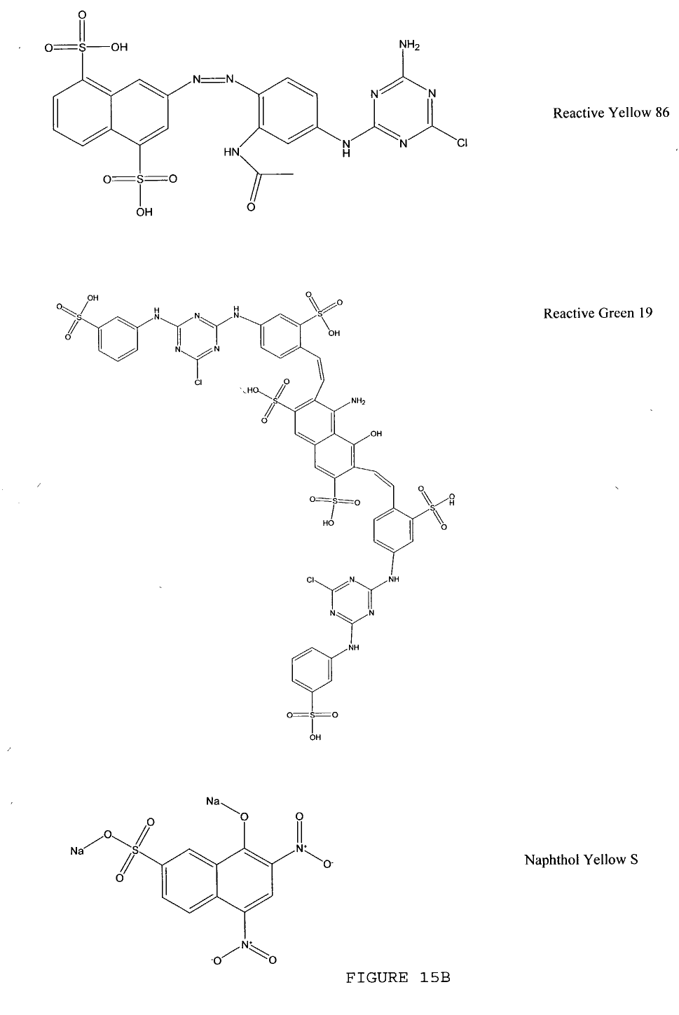

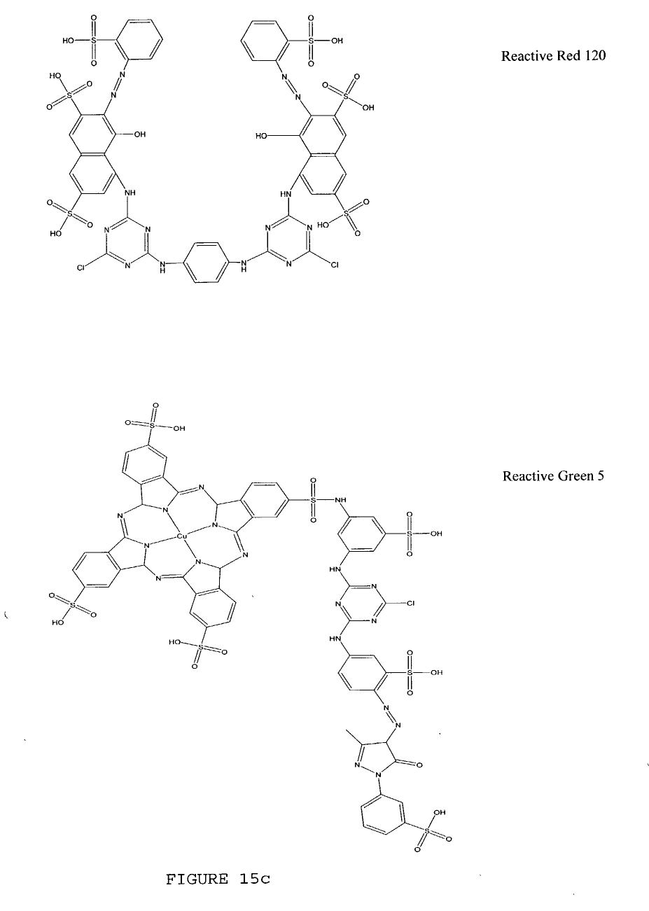

In competitive assays, the tested dyes displaced the fluorescent-based tracer FITC-NADPH, implying that the dyes bind to DOXPR at the common ligand site. A schematic representation of this displacement assay is provided in FIG. 13. In this assay, enzyme in the presence of the ligand has less FITC-NADPH bound if both of the ligands compete for the same site. At sufficiently high concentration of displacing ligand, FITC-NADPH will be completely displaced. The data for displacement of FITC-NADPH from the FITC-NADPH:DOXPR complex is provided in FIG. 14. Both Reactive Red 120 and Reactive Green 5 completely displaced FITC-NADPH from the FITC-NADPH:DOXPR complex. Calculated K_{d} values were based on curve shape and were lower than the experimentally determined IC₅₀s. Similar experiments with FITC-NADH and DHPR predict K_{d} values of 54 and 15 nM for Reactive Red 120 and Reactive Green 5, respectively.

The observed tight binding of the two dyes, RR120 and RG5, and their broad cross-reactivity across the dehydrogenase class makes them very-attractive molecules for development of displacement assays for a large number of dehydrogenases, including those of unknown function. Nonlimiting examples of suitable dehydrogenases include DHPR, DOXPR, LDH, HMGCoAR, and EACPR.

The dye molecules can be labeled to allow free and bound ligand to be distinguished. Both RR120 and RG5 have a triazinyl reactive group to which another moiety can be attached. To determine whether attachment of an additional moiety to the reactive triazinyl group of the dye will prevent it from binding to the enzymes, Sepharose columns having either RR120 or RG5 attached to them through the triazinyl group were utilized. DOXPR was able to bind to both columns, showing that modification of the reactive triazine group will not interfere with the binding. Therefore, as a first approach, attachment to the triazinyl ring is recommended. If attachment to the triazine ring proves difficult, other dyes that are similar in structure to Reactive Red 120 and Reactive Green 5 can be screened to determine which dye has an easy point of attachment.

Another attractive feature of Reactive Green 5 is the presence of chelated copper metal ion. Substitution of this metal ion with one that has fluorescence, such as ruthenium, rhenium, or osmium, will result in a ligand that can be utilized in FRET. Similarly, substitution of the chelated copper metal ion with a metal ion that has long-lifetime fluorescence, such as a lanthanide metal, particularly Eu, Tb, Dy, or Sm will result in a ligand that can be utilized in Time-Resolved FRET for all dehydrogenases that bind Reactive Green 5. Sulphophthalocyanine binds to DHPR and DOXPR. This binding suggests that the phthalocyanine moiety is responsible, or at least important, for binding. Other dyes with a sulphophthalcyanine ring system can be utilized.

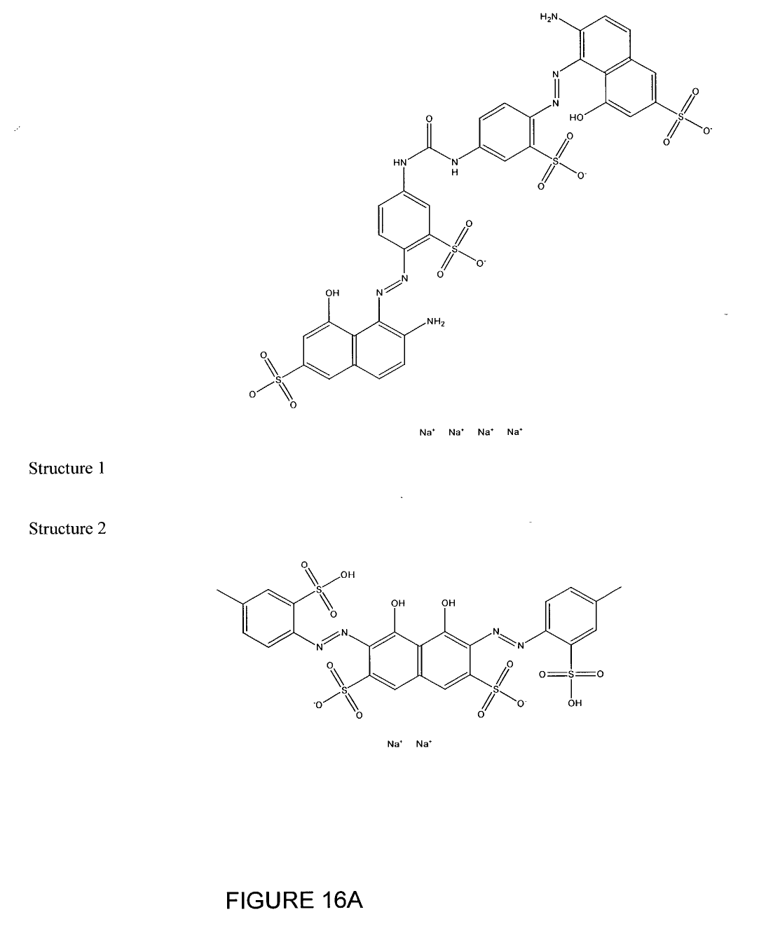

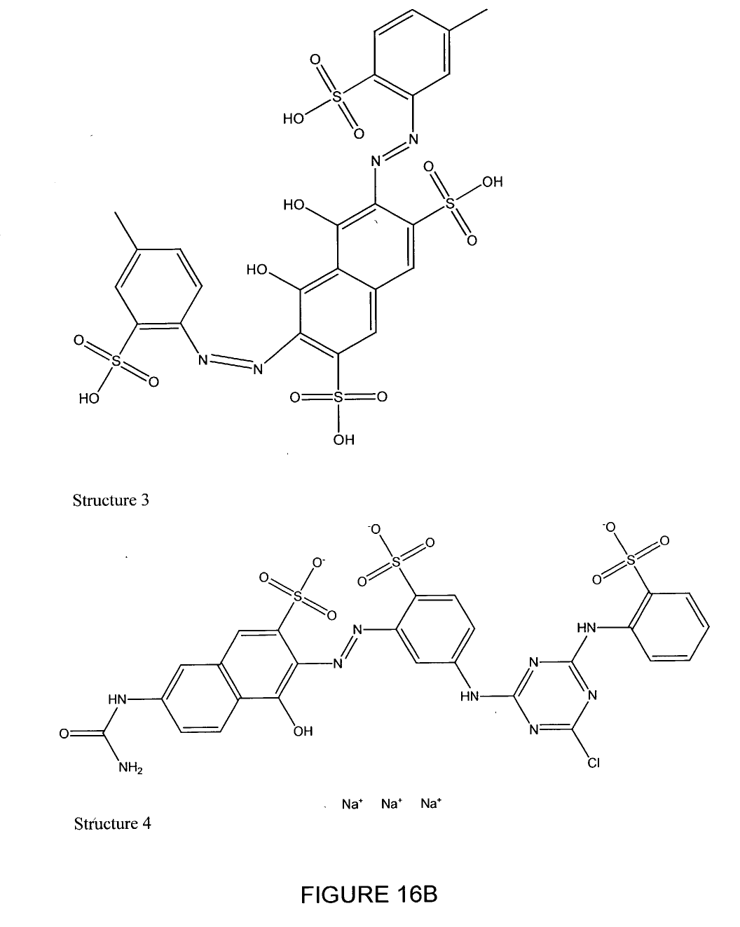

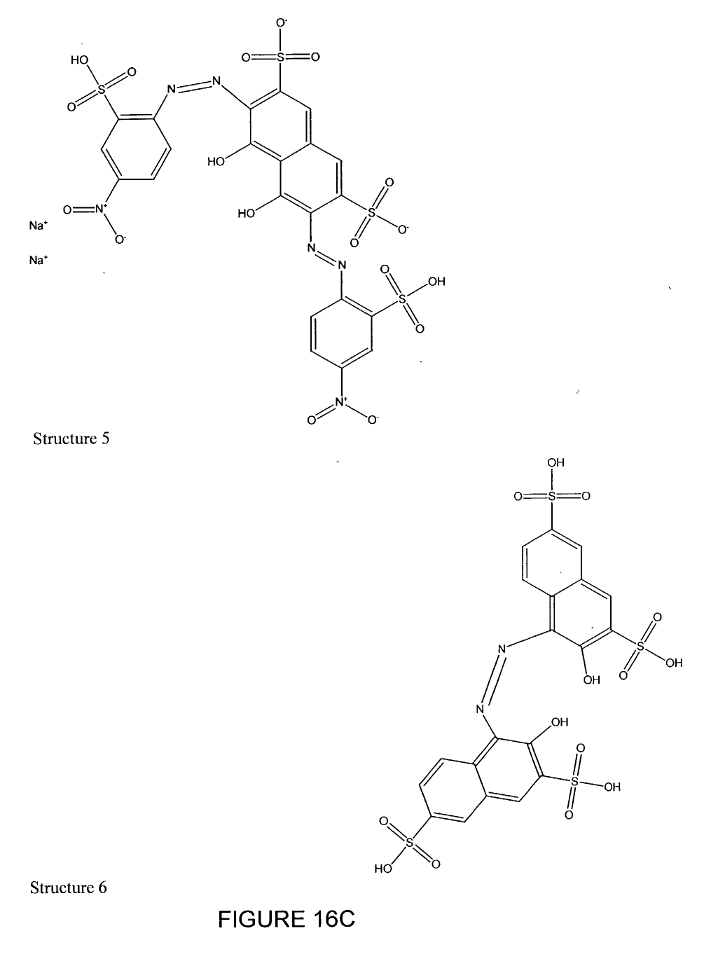

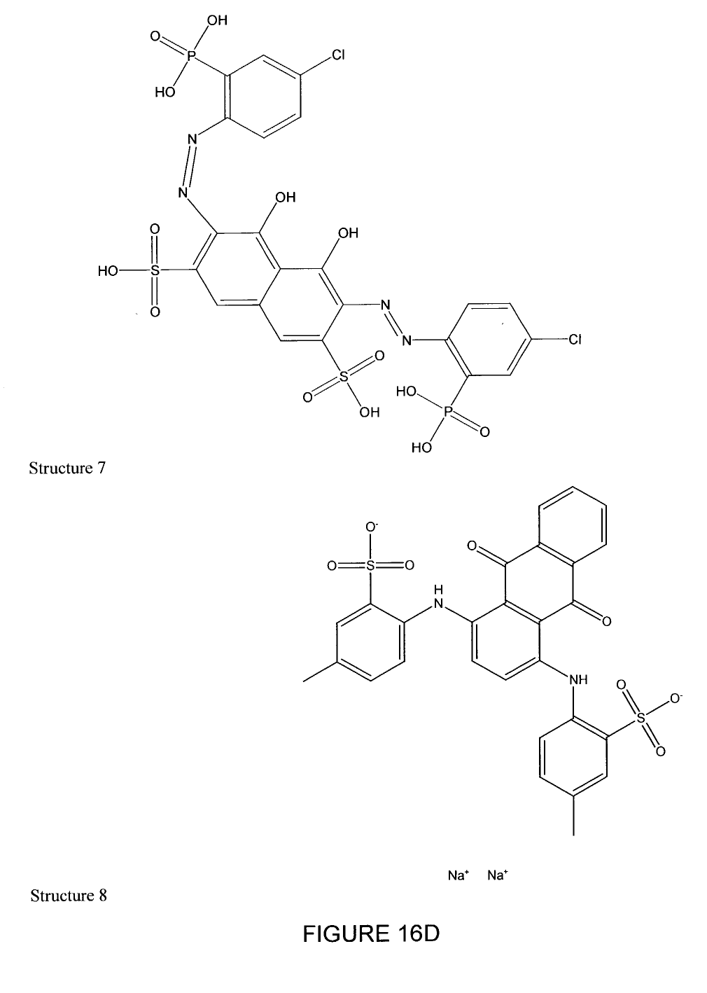

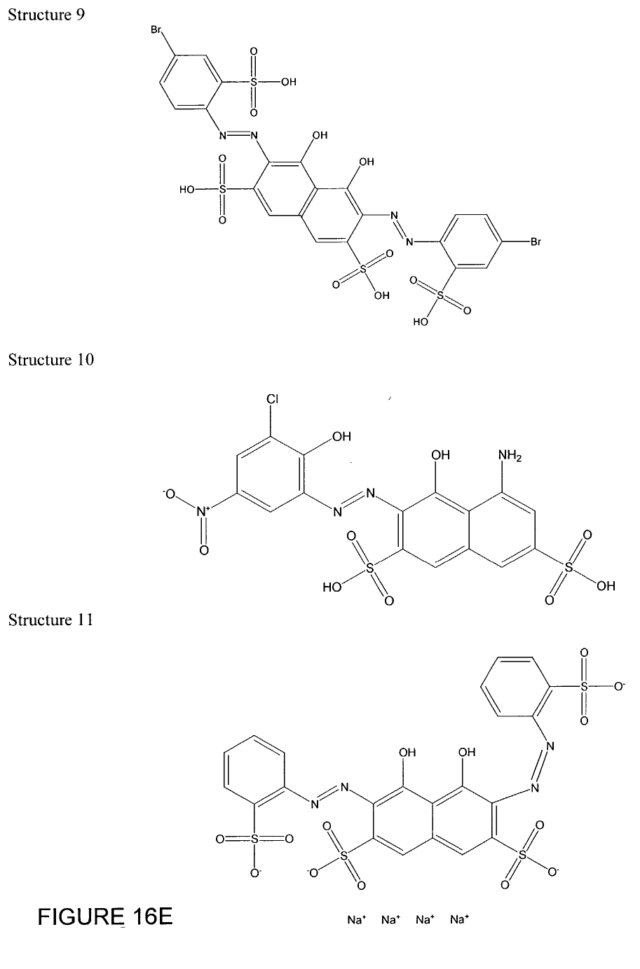

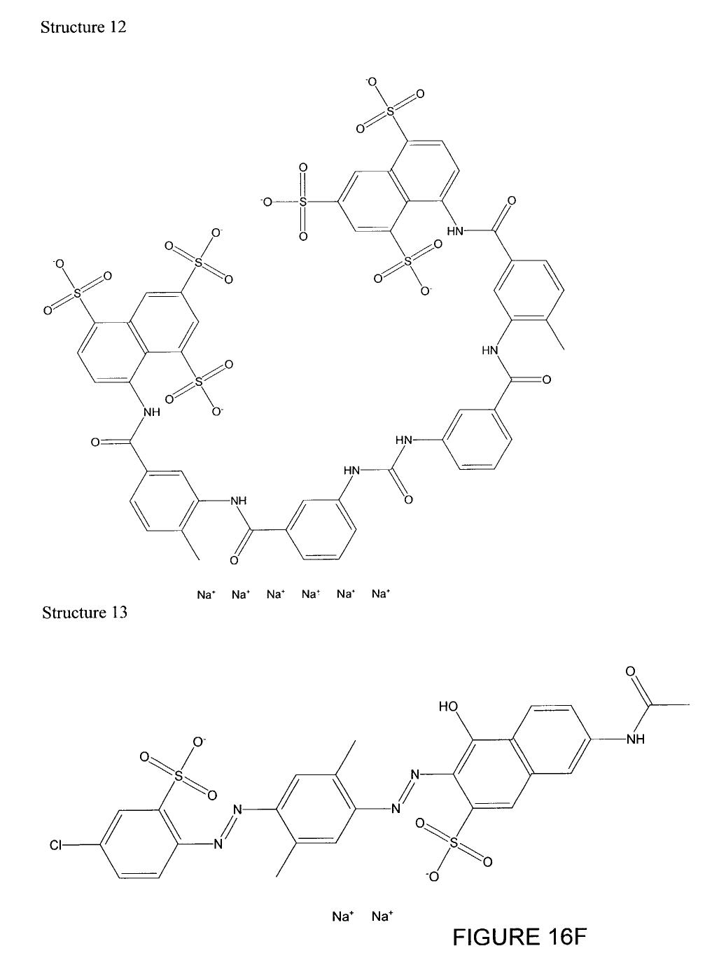

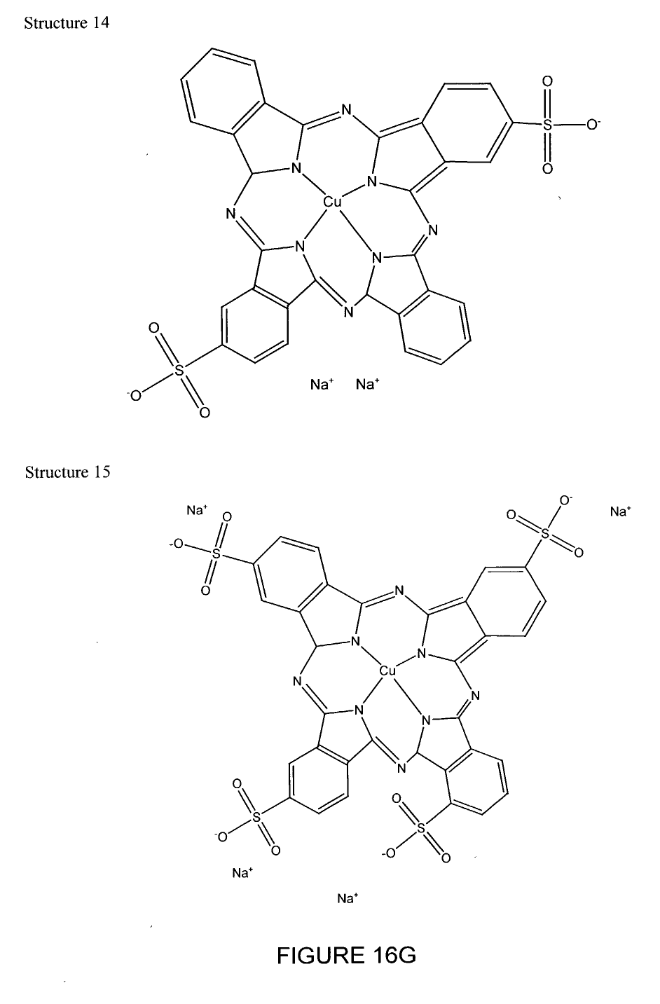

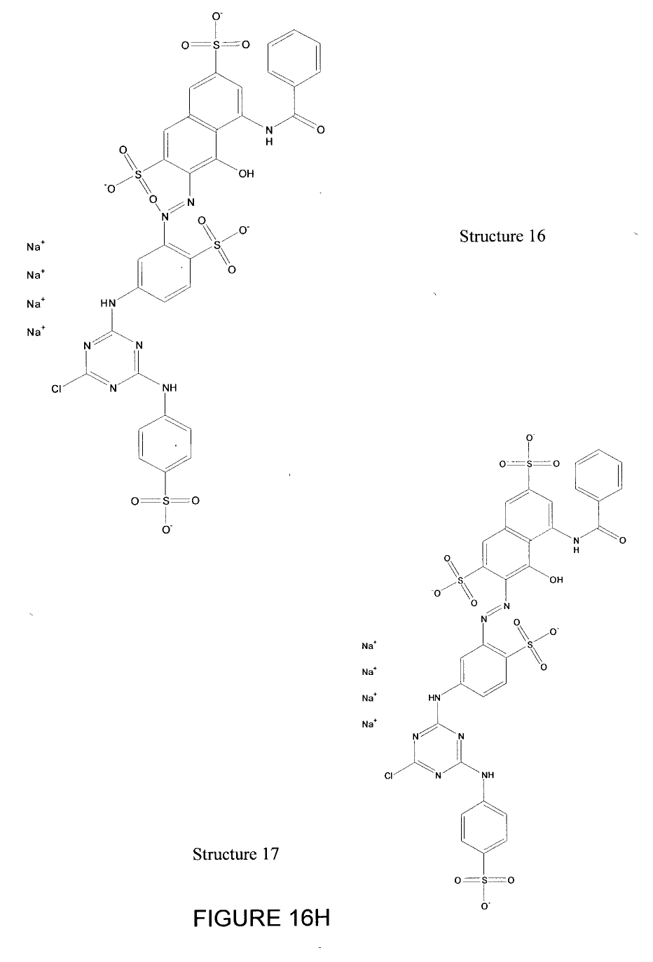

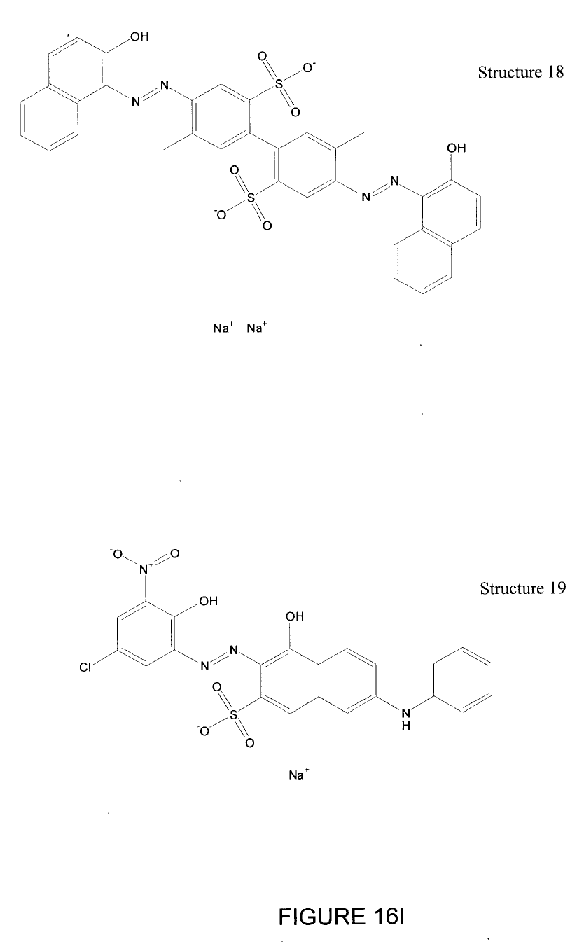

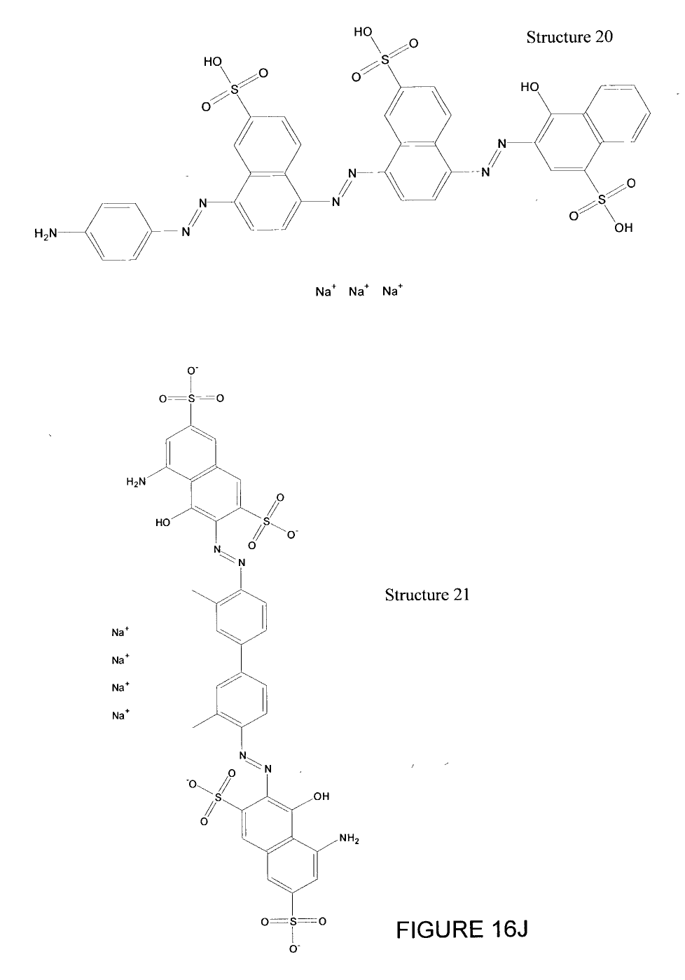

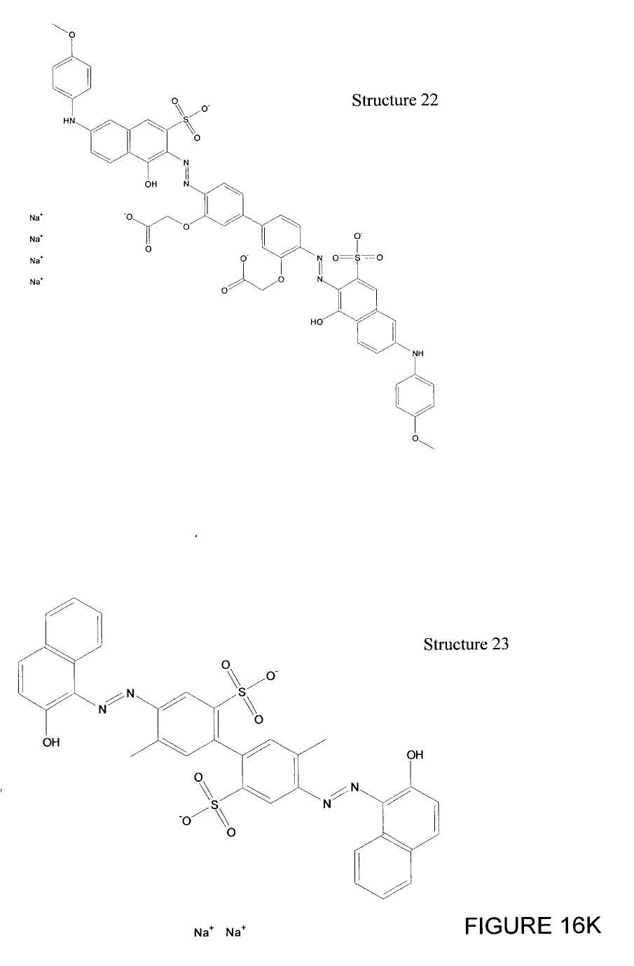

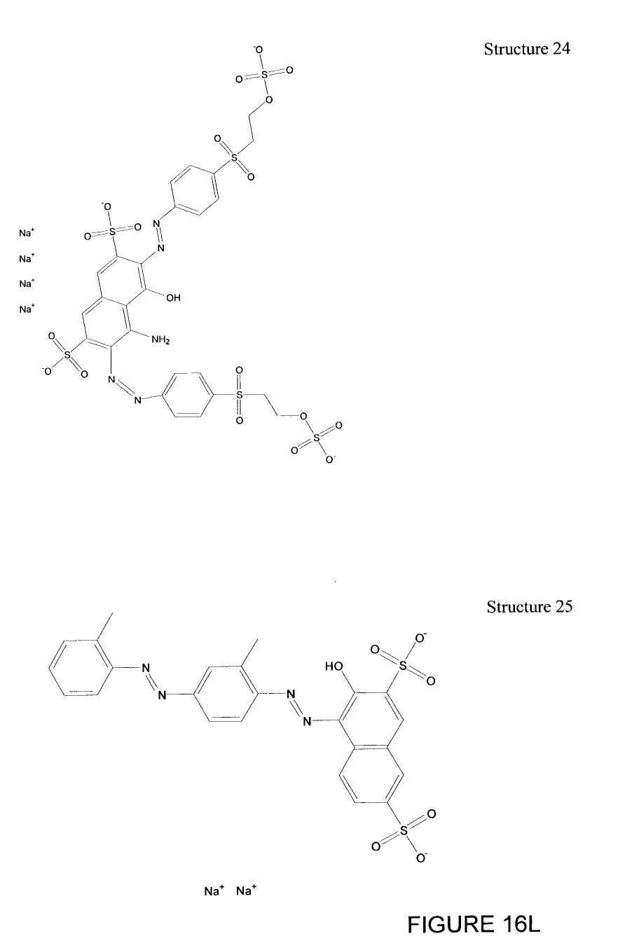

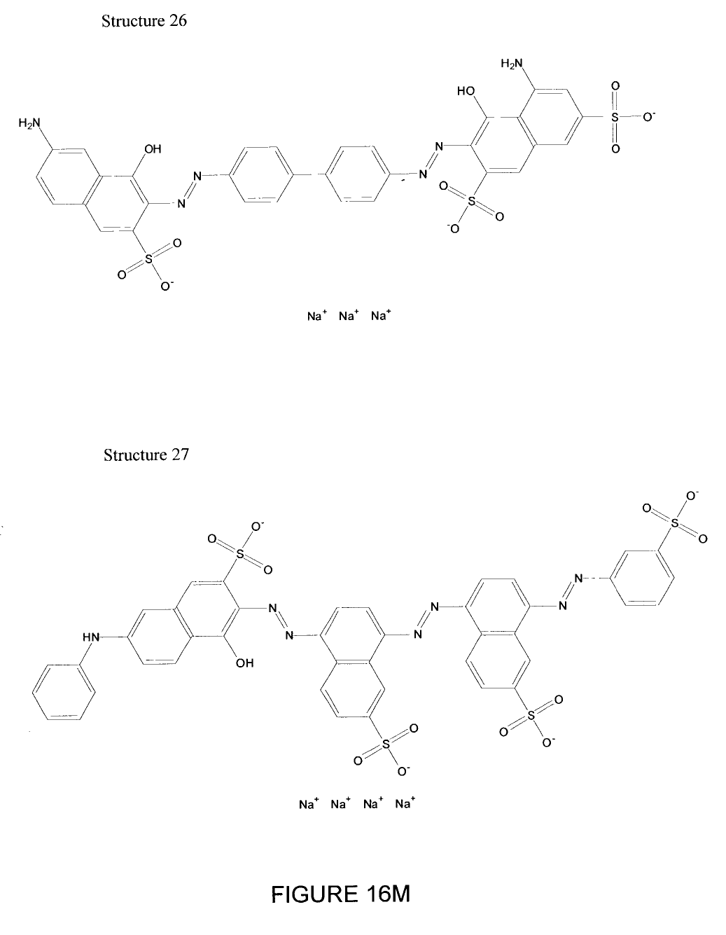

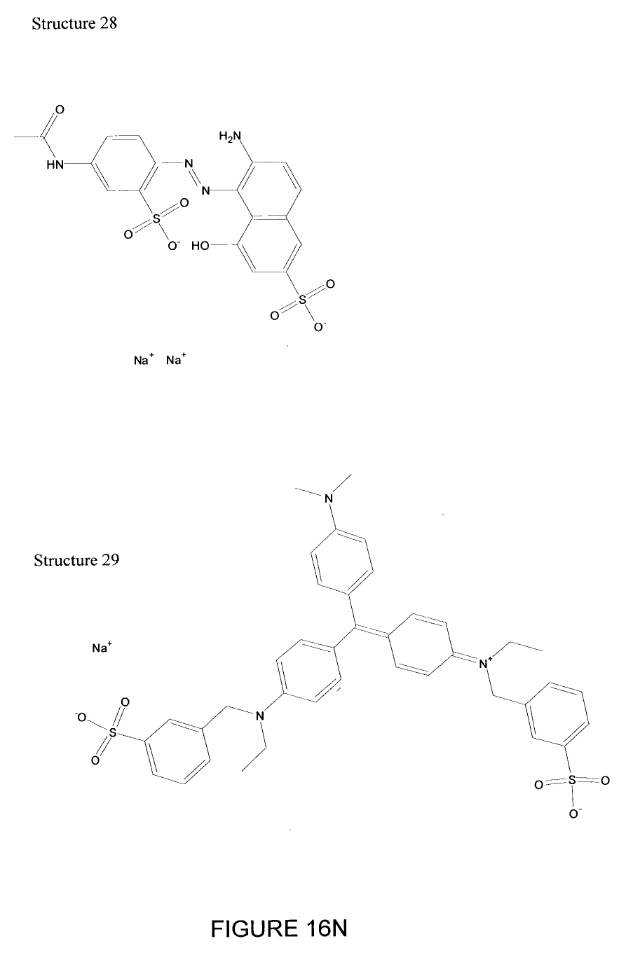

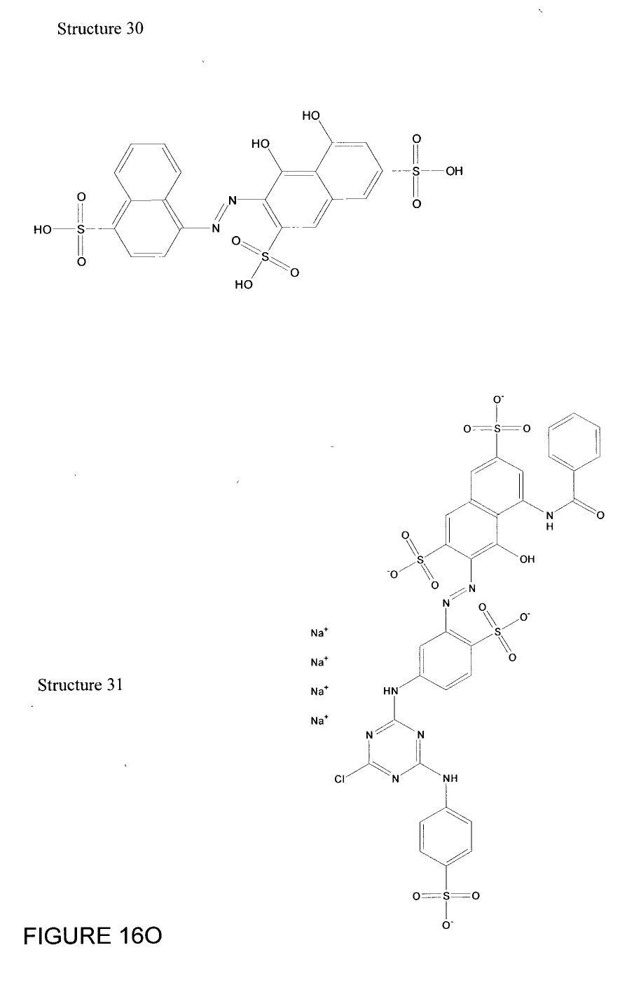

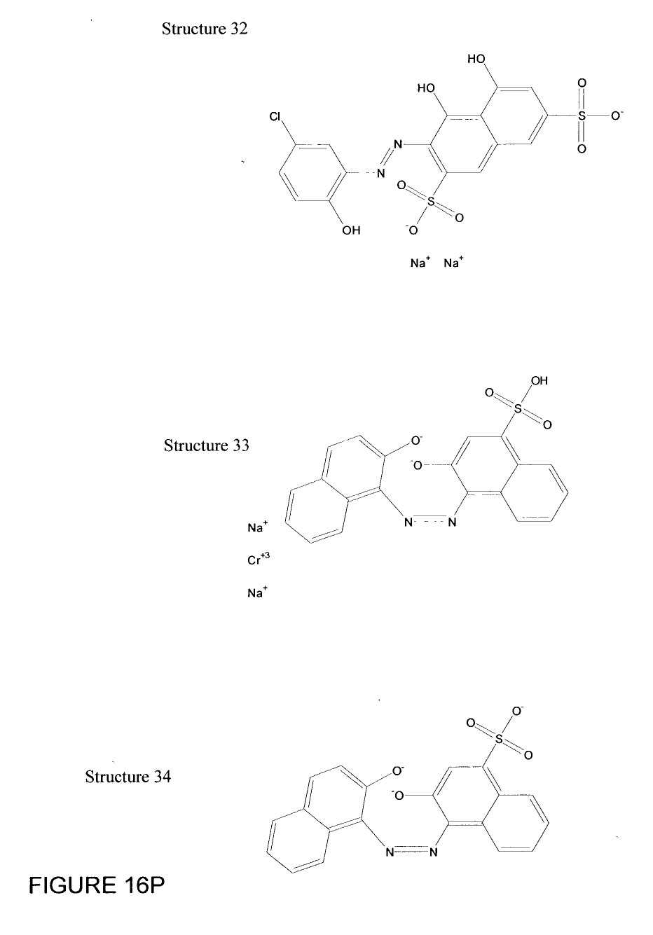

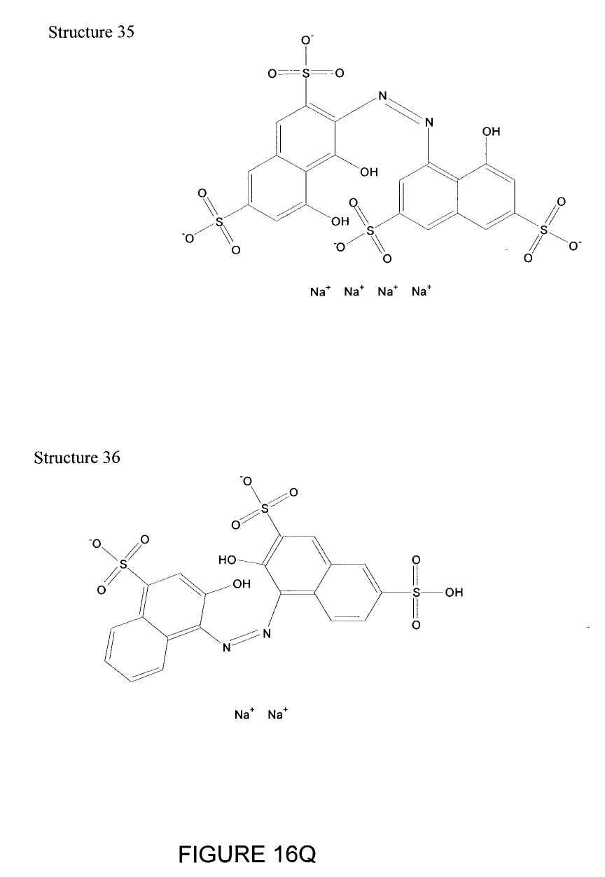

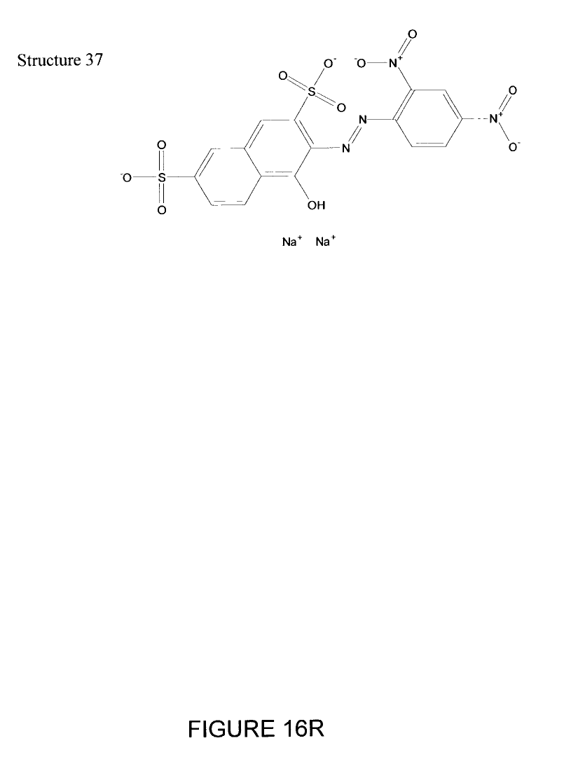

To modulate the affinity of dye binding to the target enzymes, partial molecules of dye common ligands or dye common ligands with modifications can be utilized. For example, the compounds illustrated in FIG. 16 can be utilized as common ligands in the present invention. This approach provides a broad range of targets whose affinities can be assayed using the assays of the present invention.

The present invention will make use of the observation that some dyes have preferential binding for the oxidoreductase class of enzymes with a new application in drug discovery where said well containing singular or plural compounds (ligands) could be assayed for binding activity to oxidoreductases, thus identifying novel ligands (binders) which modulate enzymatic activity of said enzymes. Coupling strategies for attaching the dyes to fluorophore tracers including and not limited to fluorescein, Cye dyes, and rhodamine green for homogeneous fluorescence based detection can be determined through Chemistry evaluation. Commercially available cages for lanthanide metals can be utilized, in place of a fluorescent molecule, to label dye molecules, resulting in time-resolved fluorescence resonance energy transfer to a second fluorophore attached or bound, for example, to the His-tag of the receptor.

Exchange strategies for replacing copper with ruthenium, rhenium, osmium, and the like can be determined through chemical evaluation, enabling development of FRET assays.

Exchange strategies for replacing copper with europium as a long lived fluorescence tracer, including all the lanthanides (Europium, Terbium, Samarium, Dysprosium, to name a few) also can be determined through chemical evaluation, enabling development of time resolved fluorescence based assays, where the dyes will be donors and acceptor molecules will be allophicocyanoprotein and not limited to rhodamine green and other small fluorophore acceptors.

### EXAMPLE 8

### Identification of Oxidoreductase Common Ligands and their Use in Displacement Assays

A population of 620 compounds were screened in the competitive displacement assay described herein. Several compounds demonstrated the ability to displace the fluorescent tracer, FITC-NAD(P)H from both DHPR and DOXPR. These compounds are depicted in FIG. 16.

It was determined that several of the common ligands identified in this experiment contain common structural motifs. For example, the compounds having structures 1 to 6 and 8 to 13 in FIG. 16 each contain the following common structural motif:

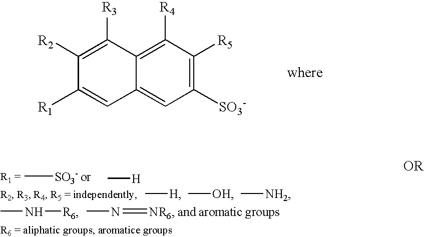

As used herein, “aromatic group” refers to a group that has a planar ring with 4n+2 pi-electrons, where n is a positive integer. Aromatic groups include heterocyclic and nonheterocyclic moieties. Nonlimiting examples of aromatic groups include benzene groups, naphthalene groups, toluene groups, xylene groups, benzyl halide groups, pyrole groups, pyrazole groups, imidazole groups, pyridine groups, pyrimidine groups, pyrazine groups, triazine groups, furan groups, oxazole groups, thiazole groups, thiophene groups, diazole groups, triazole groups, tetrazole groups, oxadiazole groups, thiodiazole groups, indole groups, benzofuran groups, benzothiophene groups, benzoimidazole groups, benzodiazole groups, benzotriazole groups, and quinoline groups.

As used herein, “aliphatic group” refers to an open-chain group or nonaromatic cyclic group. Nonlimiting examples of aliphatic groups include alkyl group, alkenyl group, and alkynyl groups. As used herein, “alkyl” means a carbon chain having from one to twenty carbon atoms. The alkyl group of the present invention can be straight chain or branched, unsubstituted or substituted. When substituted, the alkyl group can have up to ten substituent groups, such as COOH, COOAlkyl, OH, OAlkyl, OAc, SH, SO₃H, NH₂, NO₂, PH₃, PO₄H₂, H₂PO₃, H₂PO₂, CN, or X, where X is a halogen atom.

As used herein “alkenyl” means an unsaturated alkyl groups as defined above, where the unsaturation is in the form of a double bond. The alkenyl groups of the present invention can have one or more unsaturations. Nonlimiting examples of such groups include CH═CH₂, CH₂CH₂CH═CHCH₂CH₃, and CH₂CH═CHCH₃. As used herein “alkynyl” means an unsaturated alkyl group as defined above, where the unsaturation is in the form of a triple bond. Alkynyl groups of the present invention can include one or more unsaturations. Nonlimiting examples of such groups include C═CH, CH₂CH₂C≡CCH₂CH₃, and CH₂C≡CCH₃.

The presence of this common structural motif indicates that other compounds having this motif will possess binding activity to the NAD binding site of an oxidoreductase. The presence of the SO₃⁻ moiety appears to be involved in binding to oxidoreductases. The similarity of the SO₃⁻ and phosphate groups indicates that the S₃⁻ group is acting as a mimic for phosphate groups in the NAD cofactors.

A second structural motif, which is present in Reactive Green 5, was found to be present in the compounds having structures 14 and 15 in FIG. 16. This common motif, having the following structure:

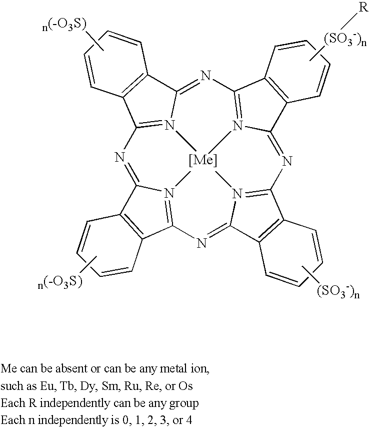

also indicates that other compounds having this motif will possess binding activity to the NAD binding site of an oxidoreductase.

An additional compound demonstrated tight binding to DHPR (k_{d}<100 nM). This compound has the following structure:

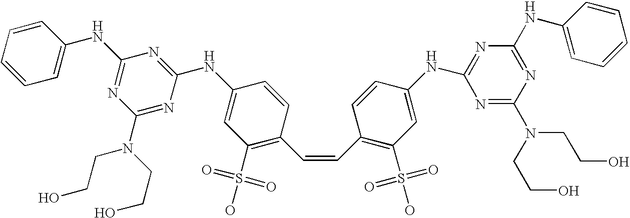

This compound demonstrated a 3-fold increase in intrinsic fluorescence upon binding to the enzyme (excitation 365 nm, emission 435 nm). It can be used either as a tracer for the screening, with or without being labeled with a detectable moiety of the invention. As such, this compound can be used for screening candidate ligands.

Each of the references and U.S. Patents cited above is hereby incorporated herein by reference.

Although the invention has been described with reference to the examples provided above, it should be understood that various modifications can be made without departing from the spirit of the invention. Accordingly, the invention is limited only by the claims.

We claim:

1. A composition comprising a common ligand linked to a detectable moiety.

2. The composition of claim 1, wherein the common ligand is an oxidoreductase common ligand.

3. The composition of claim 1, wherein the common ligand is a NAD common ligand.

4. The composition of claim 3, wherein the NAD common ligand is selected from the group consisting of NAD, NADH, NADP, and NADPH.

5. The composition of claim 3, wherein the NAD common ligand is an analog of NAD, NADH, NADP, or NADPH.

6. The composition of claim 3, wherein the NAD common ligand is a mimetic of NAD, NADH, NADP, or NADPH.

7. The composition of claim 3, wherein the detectable moiety is linked to the adenine ring of the NAD common ligand.

8. The composition of claim 3, wherein the detectable moiety is linked to the NAD common ligand via a 2-aminoethyl group.

9. The composition of claim 3, wherein the detectable moiety is linked to the N⁶ position of the NAD common ligand.

10. The composition of claim 1, wherein the common ligand is a modified NAD common ligand.

11. The composition of claim 10, wherein the NAD common ligand is modified by the addition of a 2-aminoethyl group.

12. The composition of claim 1, wherein the common ligand is a dye common ligand.

13. The composition of claim 12, wherein the dye common ligand is selected from the group consisting of Reactive Red 120, Reactive Green 5, Reactive Green 19, Reactive Blue 2, Reactive Blue 4, Reactive Blue 72, Cibacron Blue 3GA, Reactive Orange 14, Reactive Brown 10, Reactive Yellow 3, Reactive Yellow 86, and Naphthol Yellow S.

14. The composition of claim 12, wherein the dye common ligand is selected from the group consisting of Reactive Red 120, Reactive Green 5, and Reactive Blue 2.

15. The composition of claim 12, wherein the dye common ligand is Reactive Green 5 wherein a lanthanide has been substituted for the chelated copper metal ion.

16. The composition of claim 12, wherein the detectable moiety is linked to a triazine ring on the dye common ligand.

17. The composition of claim 12, wherein the detectable moiety is linked to a phthalocyanine moiety on the dye common ligand.

18. The composition of claim 1, wherein the common ligand is a compound comprising the following structural motif: 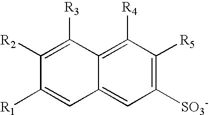
wherein R₁ is —SO₃⁻ or —H; and R₂, R₃, R₄, and R₅ each independently are selected from the group consisting of —H, —OH, —NH₂, —NH—R₆, —N═NR₆, and an aromatic group, wherein R₆ is an aliphatic or aromatic group.

19. The composition of claim 18, wherein the common ligand mimic comprises the following formula:

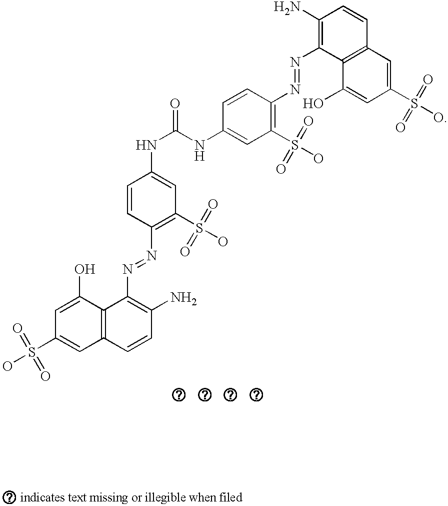

20. The composition of claim 18, wherein the common ligand mimic comprises the following formula:

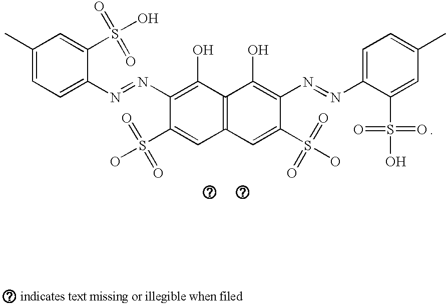

21. The composition of claim 18, wherein the common ligand mimic comprises the following formula:

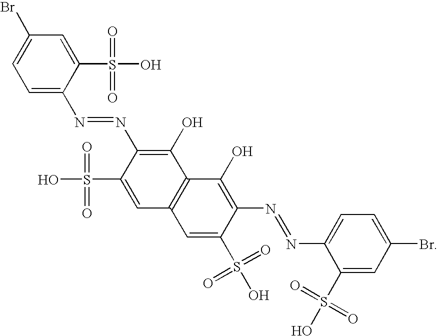

22. The composition of claim 18, wherein the common ligand mimic comprises the following formula:

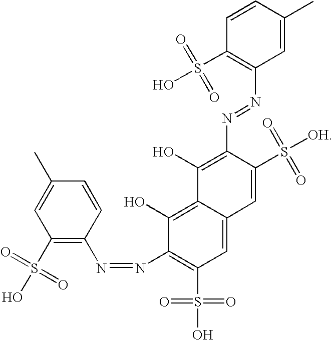

23. The composition of claim 18, wherein the common ligand mimic comprises the following formula:

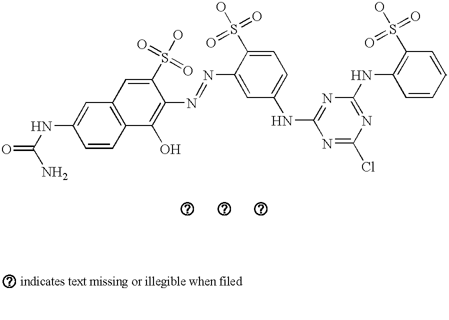

24. The composition of claim 18, wherein the common ligand mimic comprises the following formula:

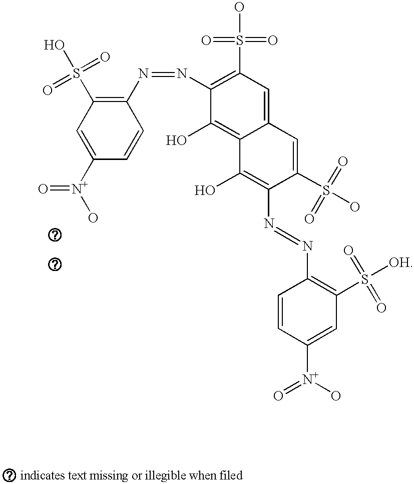

25. The composition of claim 18, wherein the common ligand mimic comprises the following formula:

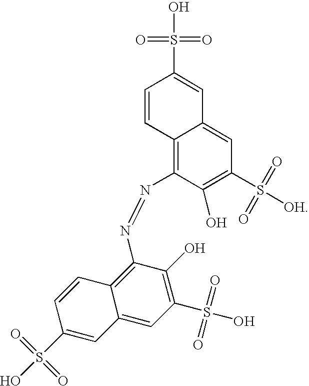

26. The composition of claim 18, wherein the common ligand mimic comprises the following formula:

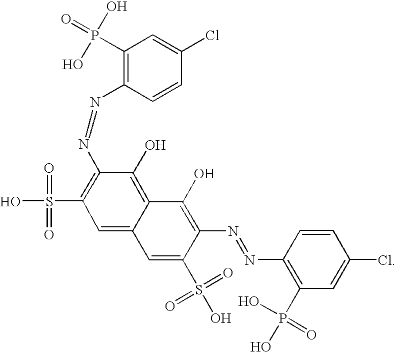

27. The composition of claim 18, wherein the common ligand mimic comprises the following formula:

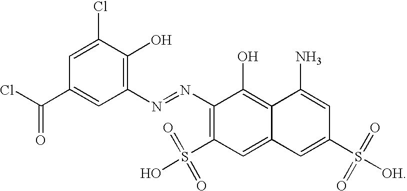

28. The composition of claim 18, wherein the common ligand mimic comprises the following formula:

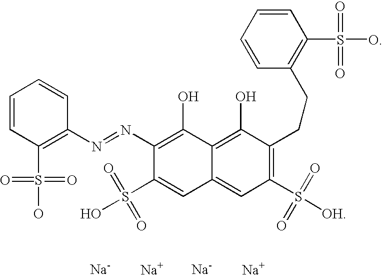

29. The composition of claim 18, wherein the common ligand mimic comprises the following formula:

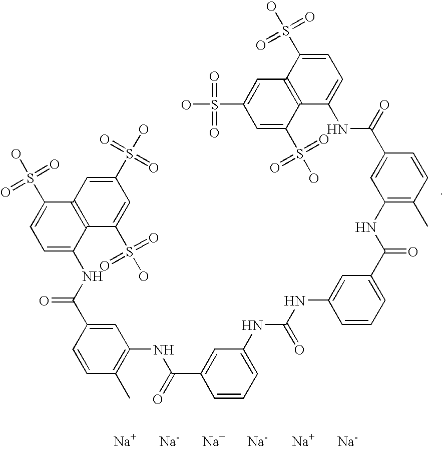

30. The composition of claim 18, wherein the common ligand mimic comprises the following formula:

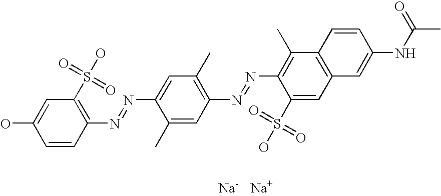

31. The composition of claim 18, wherein the common ligand mimic comprises the following formula:

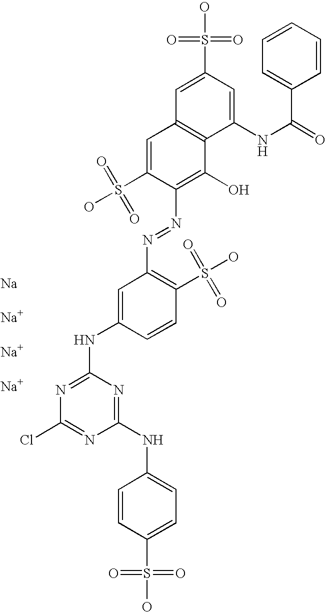

32. The composition of claim 18, wherein the common ligand mimic comprises the following formula:

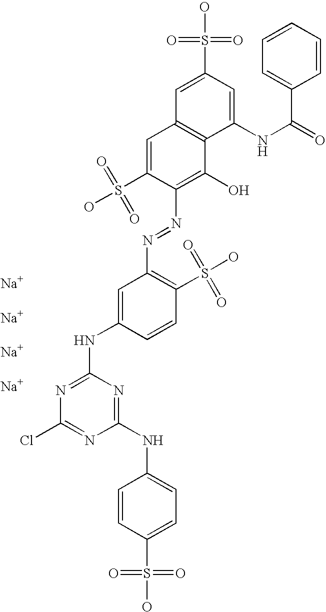

33. The composition of claim 18, wherein the common ligand mimic comprises the following formula:

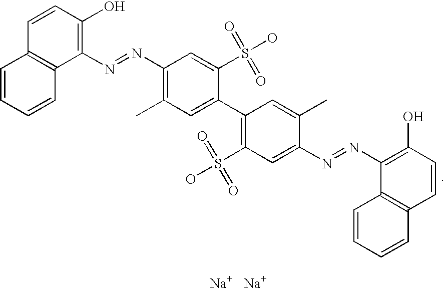

34. The composition of claim 18, wherein the common ligand mimic comprises the following formula:

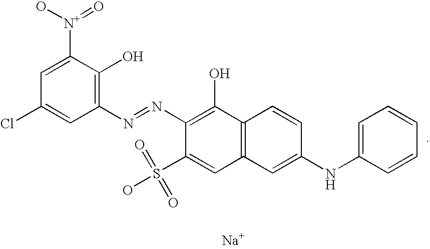

35. The composition of claim 18, wherein the common ligand mimic comprises the following formula:

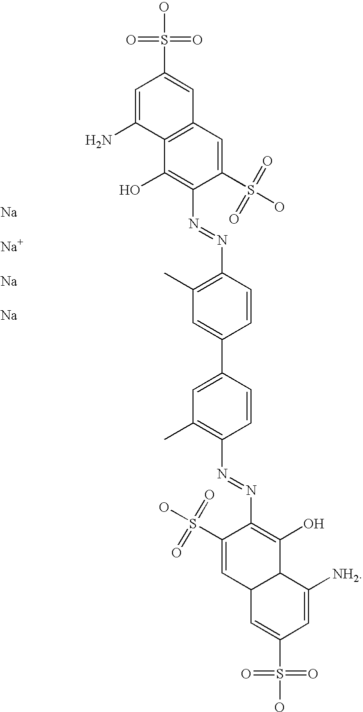

36. The composition of claim 18, wherein the common ligand mimic comprises the following formula:

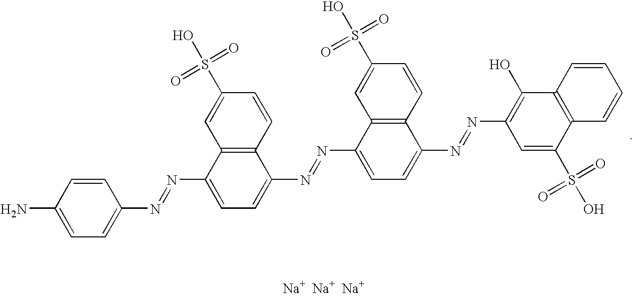

37. The composition of claim 18, wherein the common ligand mimic comprises the following formula:

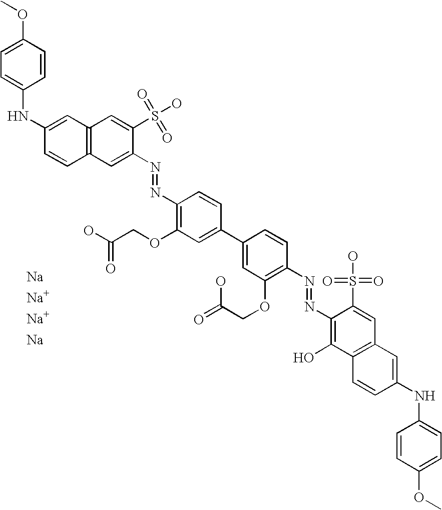

38. The composition of claim 18, wherein the common ligand mimic comprises the following formula:

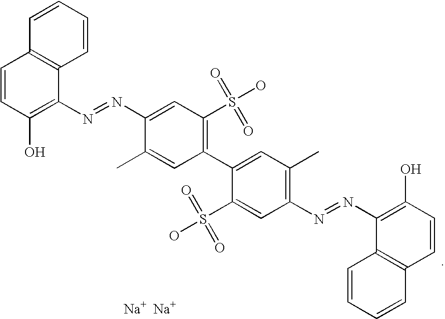

39. The composition of claim 18, wherein the common ligand mimic comprises the following formula:

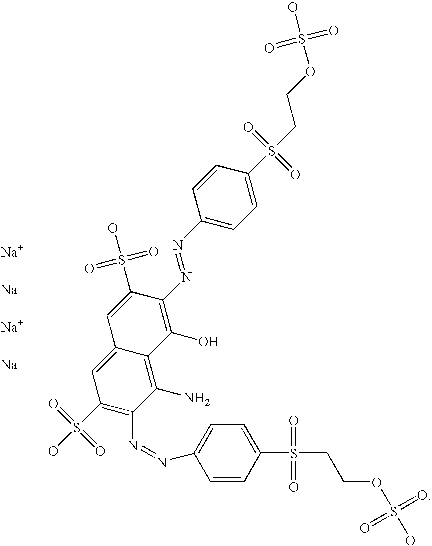

40. The composition of claim 18, wherein the common ligand mimic comprises the following formula:

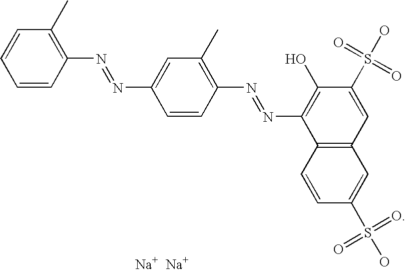

41. The composition of claim 18, wherein the common ligand mimic comprises the following formula:

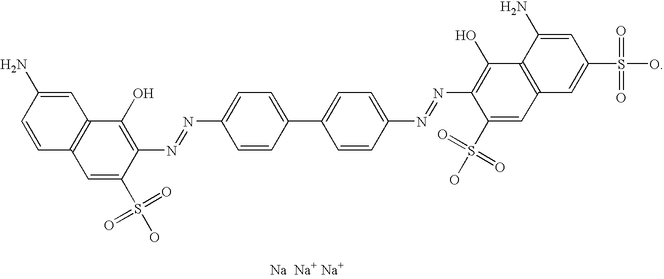

42. The composition of claim 18, wherein the common ligand mimic comprises the following formula:

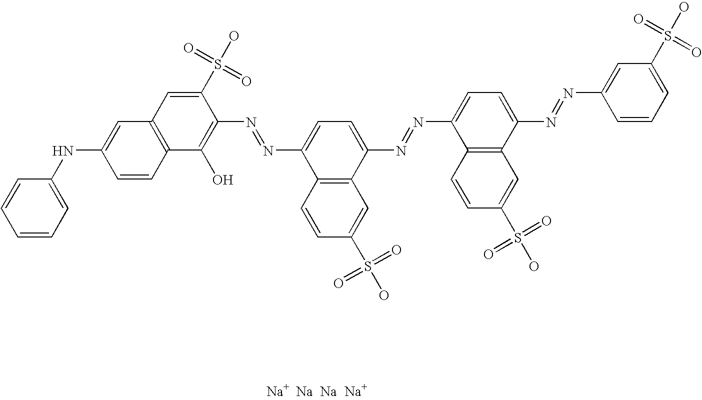

43. The composition of claim 18, wherein the common ligand mimic comprises the following formula:

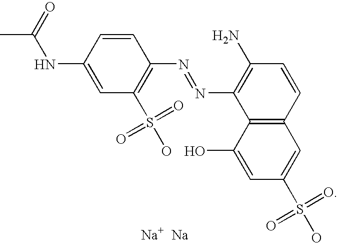

44. The composition of claim 18, wherein the common ligand mimic comprises the following formula:

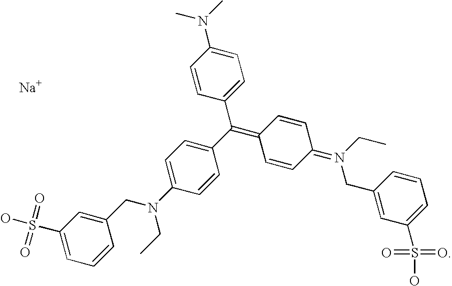

45. The composition of claim 18, wherein the common ligand mimic comprises the following formula:

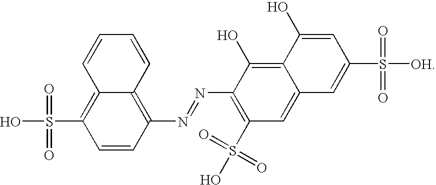

46. The composition of claim 18, wherein the common ligand mimic comprises the following formula:

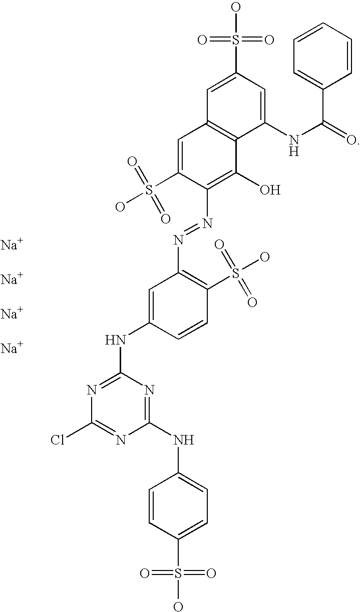

47. The composition of claim 18, wherein the common ligand mimic comprises the following formula:

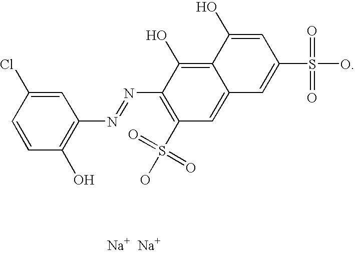

48. The composition of claim 18, wherein the common ligand mimic comprises the following formula:

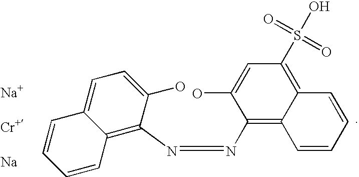

49. The composition of claim 18, wherein the common ligand mimic comprises the following formula:

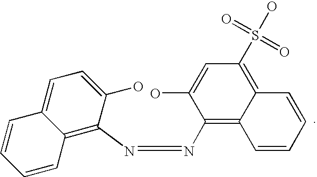

50. The composition of claim 18, wherein the common ligand mimic comprises the following formula:

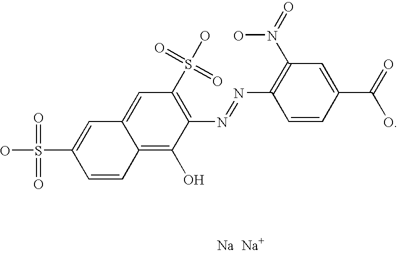

51. The composition of claim 18, wherein the common ligand mimic comprises the following formula:

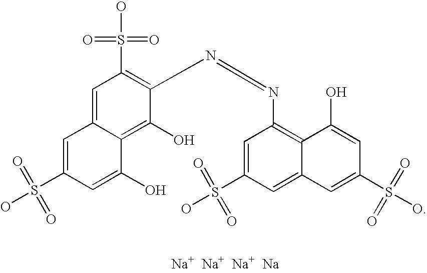

52. The composition of claim 18, wherein the common ligand mimic comprises the following formula:

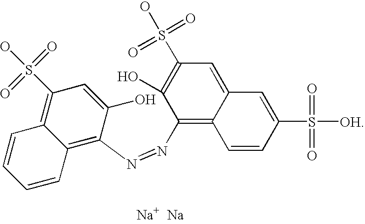

53. The composition of claim 1, wherein the common ligand is a compound comprising the following structural motif:

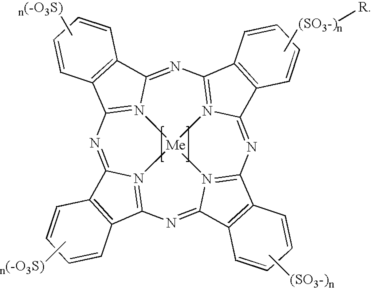

54. The composition of claim 32, wherein the common ligand mimic comprises the following formula:

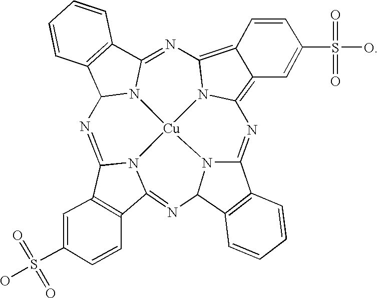

55. The composition of claim 32, wherein the common ligand mimic comprises the following formula:

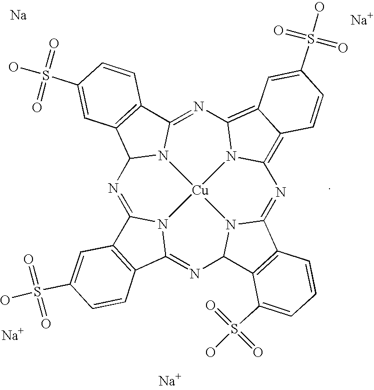

56. The composition of claim 1, wherein the common ligand mimic comprises

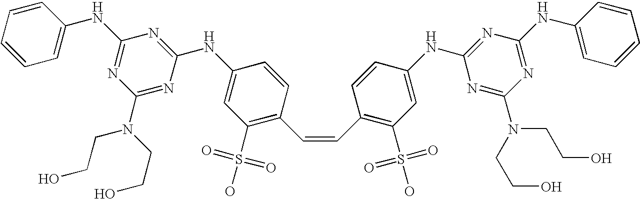

57. The composition of claim 1, wherein the common ligand mimic comprises

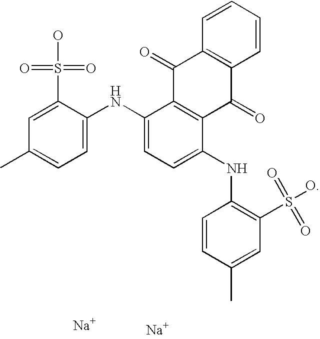

58. The composition of claim 1, wherein the detectable moiety is a fluorescent molecule.

59. The composition of claim 58, wherein the fluorescent molecule is a fluorescent protein.

60. The composition of claim 59, wherein the fluorescent protein is selected from the group consisting of green fluorescent protein and phycobiliproteins.

61. The composition of claim 58 wherein the fluorescent molecule is selected from the group consisting of fluorescein and fluorescein derivatives, rhodamine and rhodamine derivatives, BODIPY™ and BODIPY™ derivatives, Cascade Blue™ and derivatives thereof, lucifer yellow and derivatives thereof, Alexa fluor dyes, CyDye fluorescent dyes, eosins, erythrosins, Lissamine, rhodamine B, Oregon green, rhodamine green, rhodamine Red-X, Texas red and related compounds, and tetramethylrhodamine.

62. The composition of claim 58, wherein the fluorescent molecule is selected from the group consisting of fluorescein isothiocyanate, Texas Red, BODIPY™ and BODIPY™ derivatives, and CyDyes.

63. The composition of claim 58, wherein the fluorescent molecule is fluorescein isothiocyanate.

64. The composition of claim 1, wherein the detectable moiety is selected from the group consisting of radioactive tags, chromophores, ferromagnetic substances, luminescent and chemiluminescent tags, lanthanide cage metal ions, and detectable binding agents.

65. The composition of claim 64, wherein the detectable binding agent is selected from the group consisting of biotin/streptavidin, antibody/antigen, and Enzyme Acceptor/Enzyme Donor of the β-galactosidase enzyme.

66. The composition of claim 1, wherein the common ligand and detectable moiety are linked directly.

67. The composition of claim 1, wherein the common ligand and detectable moiety are linked through one or more linking group.

68. The composition of claim 1, wherein the composition is selected from the group consisting of fluorescein isothiocyano-N⁶-(2-aminoethyl)-NAD, fluorescein isothiocyano-N⁶-(2-aminoethyl)-NADH, fluorescein isothiocyano-N⁶-(2-aminoethyl)-NADP, fluorescein isothiocyano-N⁶-(2-aminoethyl)-NADPH.

69. A composition comprising a common ligand and an NAD binding receptor.

70. The composition of claim 69, wherein the NAD binding receptor is an oxidoreductase.

71. The composition of claim 70, wherein the oxidoreductase is selected from the group consisting of alcohol dehydrogenase, dihydropicolinate reductase, enoyl ACP reductase, glyceraldehyde-3-phosphate dehydrogenase, HMG CoA reductase, and lactate dehydrogenase.

72. The composition of claim 69, wherein the common ligand is an oxidoreductase common ligand.

73. The composition of claim 69, wherein the common ligand is a dye common ligand.

74. The composition of claim 73, further comprising a detectable moiety.

75. The composition of claim 69, further comprising a detectable moiety.

76. The composition of claim 75, wherein the common ligand is a NAD common ligand.

77. A method for detecting binding activity of a putative NAD binding receptor, comprising:
(a) contacting the putative receptor with a composition comprising a detectable moiety linked to a common ligand; and
(b) measuring binding of the composition to the receptor.

78. The method of claim 77, wherein unbound composition is removed prior to measurement of binding.

79. The method of claim 77, wherein the method of measuring binding is fluorescence polarization.

80. The method of claim 77, wherein the detectable moiety is a fluorescent molecule.

81. The method of claim 77, wherein the detectable moiety is a fluorescent protein.

82. The method of claim 77, wherein the detectable moiety is fluorescein isothiocyanate.

83. The method of claim 77, wherein the detectable moiety is a radioactive tag.

84. The method of claim 77, wherein the common ligand is an oxidoreductase common ligand.

85. The method of claim 77, wherein the common ligand is a NAD common ligand.

86. The method of claim 77, wherein the common ligand is a dye common ligand.

87. A method for screening candidate ligands for binding to a NAD binding receptor, comprising:
(a) contacting a NAD binding receptor with a candidate ligand and a composition comprising a common ligand linked to a detectable moiety;
(b) measuring binding of the candidate ligand to the NAD binding receptor.

88. The method of claim 87, wherein unbound candidate ligand and unbound composition are removed prior to measurement of binding.

89. The method of claim 87, wherein the method of measuring binding is fluorescence polarization.

90. The method of claim 87, wherein the detectable moiety is a fluorescent molecule.

91. The method of claim 87, wherein the detectable moiety is a fluorescent protein.

92. The method of claim 87, wherein the detectable moiety is fluorescein isothiocyanate.

93. The method of claim 87, wherein the detectable moiety is a radioactive tag.

94. The method of claim 87, wherein the common ligand is an oxidoreductase common ligand.

95. The method of claim 87, wherein the common ligand is a NAD common ligand.

96. The method of claim 87, wherein the common ligand is a dye common ligand.

97. A method for screening candidate ligands for binding to a NAD binding receptor, comprising:
(a) contacting a NAD binding receptor with a composition comprising a common ligand linked to a detectable moiety to form a composition:NAD binding receptor complex;
(b) contacting the composition:NAD binding receptor complex with a candidate ligand; and
(c) measuring displacement of the composition.

98. The method of claim 97, wherein unbound candidate ligand and unbound composition are removed prior to measurement of binding.

99. The method of claim 97, wherein the method of measuring binding is fluorescence polarization.

100. The method of claim 97, wherein the detectable moiety is a fluorescent molecule.

101. The method of claim 97, wherein the detectable moiety is a fluorescent protein.

102. The method of claim 97, wherein the detectable moiety is fluorescein isothiocyanate.

103. The method of claim 97, wherein the detectable moiety is a radioactive tag.

104. The method of claim 97, wherein the common ligand is an oxidoreductase common ligand.

105. The method of claim 97, wherein the common ligand is a NAD common ligand.

106. The method of claim 97, wherein the common ligand is a dye common ligand.

107. A method for screening candidate ligands for binding to a NAD binding receptor, comprising:
(a) contacting a NAD binding receptor with a candidate ligand to form a candidate ligand:NAD binding receptor complex;
(b) contacting the candidate ligand:NAD binding receptor complex with a composition comprising a common ligand linked to a detectable moiety; and
(c) measuring formation of a composition:NAD binding receptor complex to determine displacement of the candidate ligand.

108. The method of claim 107, wherein unbound candidate ligand and unbound composition are removed prior to measurement of binding.

109. The method of claim 107, wherein the method of measuring binding is fluorescence polarization.

110. The method of claim 107, wherein the detectable moiety is a fluorescent molecule.

111. The method of claim 107, wherein the detectable moiety is a fluorescent protein.

112. The method of claim 107, wherein the detectable moiety is fluorescein isothiocyanate.

113. The method of claim 107, wherein the detectable moiety is a radioactive tag.

114. The method of claim 107, wherein the common ligand is an oxidoreductase common ligand.

115. The method of claim 107, wherein the common ligand is a NAD common ligand.

116. The method of claim 107, wherein the common ligand is a dye common ligand.
